(19) United States
(12) Patent Application Publication
(10) Pub. No.: US 2025/0295493 A1
(43) Pub. Date: Sep. 25, 2025

(54) POST-IMPLANT ADJUSTABLE ANNULOPLASTY RING

(71) Applicant: EDWARDS LIFESCIENCES CORPORATION, Irvine, CA (US)

(72) Inventors: Rafael Pintor, Mission Viejo, CA (US); Ryan Tran Kean, Fullerton, CA (US); Hilda Z. Fann, Santa Ana, CA (US); Sai Prasad Uppalapati, Plano, TX (US); Rodolfo Rodriguez, San Luis Obispo, CA (US); Irvin John Narciso, Tustin, CA (US); Devin D. Nguyen, Garden Grove, CA (US); Taewon Kang, Irvine, CA (US); Faisal Kalam, Corona, CA (US); Louis A. Campbell, Santa Ana, CA (US); Mrunalini A. Joshi, Tustin, CA (US); William T. Biller, Tustin, CA (US); Brian S. Conklin, Orange, CA (US)

(21) Appl. No.: 19/230,828

(22) Filed: Jun. 6, 2025

Related U.S. Application Data
(63) Continuation-in-part of application No. 18/855,631, filed on Oct. 9, 2024.
(60) Provisional application No. 63/268,906, filed on Mar. 4, 2022.

(51) Int. Cl. A61F 2/24

(52) U.S. Cl. CPC A61F 2/2445; A61F 2/2466; A61F 2220/0025; A61F 2230/0095; A61F 2250/001

(57) ABSTRACT

Annuloplasty rings that are size-adjustable after implant and which facilitate deployment and reduce long-term complications. The rings each have a hollow circumference with a cinching element extending therethrough whose length is controlled remotely via a deployment system handle. The ring may be anchored to the annulus and then the size adjusted while the heart beats and under visualization to minimize regurgitation. The cinching element may be a loop of metal which is clamped to size within a junction housing to which the deployment system attaches. The rings are especially beneficial for repairing the mitral annulus, and may be saddle shaped with the junction housing located on an anterior side.

### TECHNICAL FIELD

This application is a continuation-in-part U.S. patent application Ser. No. 18/855,631, filed Oct. 9, 2024, which is a National Stage application of International Patent Application No. PCT/US2023/014440, filed Mar. 3, 2023, which claims the benefit of U.S. Patent Application No. 63/268,906, filed Mar. 4, 2022, the entire disclosures which are incorporated by reference for all purposes.

### TECHNICAL FIELD

The present disclosure relates generally to annuloplasty rings, and in particular to an adjustable mitral annuloplasty ring and deployment system.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Various surgical techniques may be used to repair a diseased or damaged valve. A commonly used repair technique effective in treating incompetence is annuloplasty, which often involves reshaping or remodeling the annulus by attaching a prosthetic annuloplasty repair segment or ring thereto. The procedure is done with the heart stopped and the patient on cardiopulmonary bypass (“on pump”). For instance, the goal of a posterior mitral annulus repair is to bring the posterior mitral leaflet forward toward to the anterior leaflet to improve leaflet coaptation. Annuloplasty rings may be stiff, flexible or semi-rigid, and a “remodeling” annuloplasty ring typically has an inner core that is “generally rigid” or “semi-rigid” in that it will flex to a small extent but resist distortion when subjected to the stress imparted thereon by the mitral valve annulus of an operating human heart.

Currently, during a mitral valve repair procedure, the size of the annuloplasty ring is determined by comparing different sizer templates to the patient's anatomy until the surgeon determines which one looks correct based on, for example, anterior leaflet area or length, intercommissural distance, and so on. However, unlike for an aortic valve replacement, where the goal is to implant the largest valve that will safely fit the patient's anatomy, mitral repair procedures implant a repair device that is somewhat smaller than the annulus to reduce the perimeter, or, more importantly, the anterior-posterior (AP) diameter, of the valve and restore leaflet coaptation. The surgeon must make an “educated guess” as to how much reduction in size is appropriate for any given patient and their specific disease state. If the wrong size repair product is chosen, the result may be a poor outcome manifested by residual mitral regurgitation (MR), insufficient coaptation length, high pressure gradients, or systolic anterior motion (SAM). If any of these conditions are found once the patient is weaned off-pump, the surgeon must make the difficult decision of going back on pump, with its associated morbidity and mortality, or leaving the patient with a sub-optimal repair, and its associated sequalae.

Given the above challenges, it would be desirable to have an annuloplasty device that could be adjusted once the patient was weaned off-pump in order to fine-tune the AP diameter (short axis) or AL-PM diameter (long axis) of the mitral valve in order to correct for small errors in the inherently imprecise sizing process. Such a ring would have the potential to reduce poor mitral valve repair outcomes and the need to go back on-pump in many cases to address them. Once adjustments were made and desired outcome achieved, the deployment system attachments could be disengaged, leaving the patient with a customized annuloplasty device that was tailored to their specific anatomy.

In attempts to vary the shape of the repair device, adjustable annuloplasty devices such as the Cardinal mitral annuloplasty system originally from Valtech Ltd. of Israel, now a part of Edwards Lifesciences of Irvine, CA. The Cardinal system has a semi-rigid annuloplasty ring enabled for ring diameter fine tuning and optimization of leaflet coaptation on a beating heart under real-time echocardiographic guidance. The Cardinal system is disclosed in U.S. Pat. Nos. 8,241,351 and 10,363,136 which are expressly incorporated herein by reference for all purposes.

Despite past attempts, there is a need for an annuloplasty ring that may be shaped adjusted to effect repair of a malfunctioning valve while avoiding negative outcomes.

### SUMMARY

Disclosed here are adjustable annuloplasty rings and deployment systems. The annuloplasty rings are size-adjustable after implant and facilitate delivery and reduce long-term complications. The rings each have a hollow circumference with a cinching element extending therethrough whose length is controlled remotely via a deployment system handle. The ring may be anchored to the annulus and then the size adjusted while the heart beats and under visualization to minimize regurgitation. The cinching element may be a loop of metal which is clamped to size within a junction housing to which the deployment system attaches.

The rings are especially beneficial for repairing the mitral annulus, and may be saddle shaped with the junction housing located on an anterior side or posterior side. Each ring is advanced down to the mitral annulus along anchoring sutures. A distal end of a delivery or deployment system attaches to a junction housing at a midpoint of an anterior segment of the annuloplasty ring and in conjunction with a semi-rigid ring holder handle is used to parachute the ring down the array of sutures. The deployment system has a shaft long enough to extend from outside the body to the surgical site, and is connected to a proximal control handle. The length of the shaft allows for partial/full sternotomy and right lateral thoracotomy procedures.

A first exemplary annuloplasty ring and shape adjustment system disclosed herein, comprises an annuloplasty ring defining a continuous peripheral shape around a central aperture and central axis. The annuloplasty ring has a contractible inner body defining a lumen extending therethrough, wherein the inner body extends around the peripheral shape and has two ends connected to each other by a junction housing. The annuloplasty ring further includes a flexible non-extensible cinching element formed in a complete loop, the cinching element passing through the inner body lumen and into the junction housing. The junction housing has an inner cavity with three openings—first and second aligned openings in communication with the inner body lumen at the two ends of the inner body, and a third opening at an upper port facing axially up from the housing. The cinching element has a main portion generally in a plane of the inner body and a subloop that projects perpendicular to the main portion through the upper port. Finally, the inner body has a relaxed implant shape when the cinching element is under no tension and a smaller, contracted shape when the cinching element is placed under tension.

The first system junction housing may have external threads for attachment of a delivery shaft thereto. The annuloplasty ring may further include a locking member within the junction housing configured to clamp onto the cinching element and maintain the cinching element under tension around the inner body. For instance, the locking member comprises a locking screw positioned in a vertical channel of the junction housing aligned with the upper port and having external threads which engage internal threads defined within the vertical channel. The locking screw has a non-circular internal throughbore that receives the subloop of the cinching element, wherein rotational advancement of the locking screw down into the vertical channel eventually clamps divergent portions of the cinching element against the inner cavity of the junction housing.

A second annuloplasty ring and shape adjustment system disclosed herein comprises an annuloplasty ring defining a continuous peripheral shape around a central aperture and a central axis. The annuloplasty ring has a contractible inner body defining a lumen extending therethrough, wherein the inner body extends around the peripheral shape and has two ends connected to each other by a junction housing. The annuloplasty ring further includes a flexible cinching element formed in a continuous loop, the cinching element passing through the inner body lumen and into the junction housing. The cinching element forms a subloop through a port formed in the junction housing, and the inner body has a relaxed implant shape when the cinching element is under no tension and a smaller, contracted shape when the cinching element is placed under tension. The second system further includes a deployment system including an elongated deployment shaft having a distal end configured to attach to the port of the junction housing, and a proximal control handle. The control handle has an adjustment cable connected to a tension actuator configured to pull the adjustment cable in a proximal direction. The adjustment cable has a length sufficient to extend through a lumen in the elongated shaft and loop through the subloop. Actuation of the tension actuator pulls the adjustment cable and in turn places the cinching element under tension to convert the inner body from the relaxed implant shape toward the smaller, contracted shape.

In the second system, the annuloplasty ring further may include a locking member within the junction housing configured to clamp onto the cinching element and maintain the cinching element under tension around the inner body. Further, the control handle may have a locking actuator connected to an elongated torque shaft configured to slide axially and rotate concentrically within the deployment shaft. The torque shaft has a distal non-circular torque driver shaped to mate with a similarly-sized and shaped opening in the locking member, wherein rotation of the torque shaft causes the locking member to clamp onto the cinching element.

In the second system, the locking member may comprise a locking screw positioned in a vertical channel of the junction housing aligned with the port. The locking screw may have external threads which engage internal threads defined within the vertical channel. The locking screw also has a throughbore that receives the subloop of the cinching element. The torque shaft and torque driver are hollow to receive the subloop and the adjustment cable passes through the torque shaft to loop through the subloop. Consequently, rotational advancement of the locking screw down into the vertical channel eventually clamps divergent portions of the cinching element against the inner cavity of the junction housing.

The second system tension actuator and locking actuator may be wheels positioned within a housing of the control handle and mounted to rotate about a longitudinal axis of the housing. The wheels may be exposed through windows formed in the housing with each having a locking collar associated therewith with a tab extending to the exterior of the housing, the tabs being arranged to toggle between permitting and preventing rotation of a respective wheel. The wheels may each have a scalloped or uneven outer surface to facilitate rotation.

In the second system, the junction housing may have external threads for attachment of the delivery system thereto, and the control handle has an attachment actuator connected to an elongated attachment shaft configured to slide axially and rotate concentrically within the deployment shaft. The attachment shaft also has a distal end with internal threads for mating with the external threads on the junction housing, wherein rotation of the attachment shaft enables attachment and detachment of the delivery system from the annuloplasty ring.

Alternatively, the second system delivery system may have a quick-release harness in the form of a loop of flexible material that extends from the control handle and around the junction housing, wherein one end of the loop of flexible material is severable within the control handle to enable the detachment of the delivery system from the annuloplasty ring.

In either the first or second system, the cinching element is metallic, such as a cobalt chromium alloy. The inner body may be formed of a metallic tube having V-shaped gaps formed therein to permit contraction. The inner body may be heat set into a saddle shape, and may be nitinol. Further, the annuloplasty ring in either system may include a hollow compressible filler member extending within the inner body lumen and through which the cinching element extends. The ring may also have a tubular fabric cover surrounding the inner body and having a sewing cuff of a ring of fabric attached to an outer periphery of the fabric cover.

A third annuloplasty ring and shape adjustment system comprises an annuloplasty ring defining a continuous peripheral shape around a central aperture and a central axis. The annuloplasty ring has a contractible inner body defining a lumen extending therethrough, wherein the inner body extends around the peripheral shape and has two ends connected to each other by a junction housing. The annuloplasty ring further includes a flexible cinching element formed in a continuous loop, the cinching element passing through the inner body lumen and into the junction housing. The junction housing has an inner cavity opening to an upper port, wherein the cinching element forms a subloop through the upper port. The inner body has a relaxed implant shape when the cinching element is under no tension and a smaller, contracted shape when the cinching element is placed under tension. A deployment system includes an elongated deployment shaft having a distal end configured to attach to the upper port of the junction housing, the deployment system being configured to apply variable tension to the subloop of the cinching element. The annuloplasty ring further includes at least one finger spring-biased toward the junction housing, wherein the finger is positioned and sized to contact a portion of the subloop extending out of the junction housing and trap the portion against the junction housing.

A fourth annuloplasty ring and shape adjustment system comprises an annuloplasty ring defining a continuous peripheral shape around a central aperture and a central axis. The annuloplasty ring has a contractible inner body defining a lumen extending therethrough. The inner body extends around the peripheral shape and has two ends connected to each other by a junction housing. The annuloplasty ring further including a flexible cinching element formed in a continuous loop, the cinching element passing through the inner body lumen and into the junction housing. The junction housing has an inner cavity opening to an upper port, wherein the cinching element forms a subloop through the upper port. The inner body has a relaxed implant shape when the cinching element is under no tension and a smaller, contracted shape when the cinching element is placed under tension. A locking member within the junction housing is configured to clamp onto the cinching element and maintain the cinching element under tension around the inner body. Also, the locking member is adapted to apply a clamping force calibrated to resist expansion of the annuloplasty ring from natural cardiac cycling motion, but which enables the cinching element to slip through the locking member from expansion forces associated with balloon expansion from within the annuloplasty ring.

A still further fifth annuloplasty ring and shape adjustment system comprises an annuloplasty ring defining a continuous peripheral shape around a central aperture and a central axis. The annuloplasty ring having a contractible inner body defining a lumen extending therethrough, wherein the inner body extends around the peripheral shape and has two ends connected to each other by a junction housing. The annuloplasty ring further including a flexible cinching element formed in a continuous loop, the cinching element passing through the inner body lumen and into the junction housing. The junction housing has an inner cavity opening to an upper port, wherein the cinching element forms a subloop through the upper port. The inner body has a relaxed implant shape when the cinching element is under no tension and a smaller, contracted shape when the cinching element is placed under tension. Also, a locking member within the junction housing is configured to clamp onto the cinching element and maintain the cinching element under tension around the inner body. The cinching element is strong enough to resist breakage from natural cardiac cycling motion, but is calibrated to break from expansion forces associated with balloon expansion from within the annuloplasty ring. Further, a secondary cord formed in a continuous loop passes through the inner body lumen. The secondary cord has a longer length than the circumference of the inner body and is strong enough to resist breakage from forces associated with balloon expansion from within the annuloplasty ring to enable limited post-implant expansion of the annuloplasty ring with a balloon.

A further understanding of the nature and advantages will become apparent by reference to the remaining portions of the specification and drawings.

### BRIEF DESCRIPTION OF THE DRAWINGS

Features and advantages of the present disclosure will become appreciated as the same become better understood with reference to the specification, claims, and appended drawings wherein:

FIG. 1A-1E are perspective views of an atrial side of a mitral annulus illustrating several steps of implanting and adjusting an annuloplasty ring of the present application;

FIG. 2 is a perspective view of an exemplary deployment system for the adjustable annuloplasty ring, and FIG. 2A is an enlarged view of the distal end of a deployment shaft coupled to the annuloplasty ring;

FIG. 3 is a perspective view of the distal end of the deployment shaft shown in phantom engaged with a junction housing of the annuloplasty ring;

FIG. 4A is a perspective view of an inner coupling member of the deployment shaft engaged with the ring junction housing, and FIG. 4B shows disengagement of the coupling member from the junction housing;

FIG. 5A is a vertical sectional view of the interaction between the distal end of the deployment shaft and the junction housing of the annuloplasty ring, FIG. 5B is an exploded perspective view of the components thereof, and FIG. 5C (sheet 12) is a vertical sectional view showing tension being applied by a tension adjustment cable through the deployment shaft to a cinching element within the annuloplasty ring;

FIG. 6 is a perspective view of the exemplary adjustable annuloplasty ring, FIG. 6A is an enlarged cutaway view of one portion thereof, and FIG. 6B is a radial sectional view thereof;

FIG. 7 is a perspective view of the distal end of the deployment shaft coupled to the adjustable annuloplasty ring having an outer cover removed, and FIG. 7A is an exploded perspective view of the inner components of the annuloplasty ring;

FIG. 8A is a perspective view of the adjustable annuloplasty ring with the outer cover and an inner filler tube removed, and FIG. 8B is the same perspective view with a coiled body removed illustrating the cinching element;

FIG. 9A is an enlarged perspective view of a junction housing of the adjustable annuloplasty ring, and FIG. 9B is a cutaway view of the junction housing showing internal components thereof;

FIG. 10A is a view similar to FIG. 9B showing tension being applied by the adjustment cable to the cinching element, FIG. 10B is a view of the junction housing being acted on by a locking driver of the deployment system, and FIG. 10C shows the junction housing after the cinching element has been locked in place;

FIG. 11 is a perspective view of a proximal control handle of the deployment system with a front cover removed to illustrate certain internal components, including an enlarged, exploded view of a distal end of the deployment shaft and inner components;

FIG. 12 is an exploded perspective view of the proximal control handle of the deployment system, and FIG. 12A is an enlarged view of the connection between the tension adjustment cable and control handle;

FIG. 13A-13C are perspective views of the proximal control handle during operation thereof, and FIG. 13D is an enlarged view of an operation to sever the adjustment cable within the deployment system;

FIG. 14 is a schematic view of an anterior segment of the adjustable annuloplasty ring without a sewing cuff and with an outer cover partially removed and a suture being threaded through an outer periphery thereof, and FIG. 14A is a sectional view through the annuloplasty ring indicating one possible path of the suture through the ring;

FIG. 15 is a sectional view through an annuloplasty ring of the present application having an outer sewing cuff and indicating one possible path of an anchoring suture through the ring;

FIGS. 16A-16F are radial sectional views through alternative annuloplasty rings showing a variety of supplemental sewing cuffs that can be used;

FIG. 17 is a front elevational view of a saddle-shaped coiled body that may be used with the adjustable annuloplasty rings of the present application;

FIG. 18 is a front elevational view of an alternative saddle-shaped coiled body that may be used with the adjustable annuloplasty rings of the present application;

FIG. 19A is a front elevational view of a still further saddle-shaped coiled body, and FIG. 19B schematically illustrates expansion of the coil body during expansion of the annulus during the normal cardiac cycle;

FIG. 20 is a front elevational view of another saddle-shaped coiled body for an annuloplasty ring having a size adjustable remodeling core positioned therethrough, and

FIG. 21 is a front elevational view of the remodeling core;

FIG. 22 is a view of the inner components of the saddle-shaped annuloplasty ring similar of FIG. 20 with the addition of a cinching element, and

FIG. 23 is a sectional view through a junction housing thereof;

FIG. 24 is a front elevational view of inner components of a still further saddle-shaped annuloplasty ring having a shaped sleeve component surrounding a posterior segment to form an upward bow, and FIG. 24A is a sectional view through the posterior segment thereof;

FIG. 25 is a front elevational view of inner components of another saddle-shaped annuloplasty ring having a shaped sleeve component surrounding an anterior segment to form an upward bow;

FIG. 26 is a front elevational view of a saddle-shaped support body for an annuloplasty ring formed by laser cutting a nitinol tube and a junction housing;

FIG. 27 is a perspective view of a junction housing for the adjustable annuloplasty rings described herein illustrating one potential drawback of using an elongated cinching element;

FIG. 28A is a perspective view of a junction housing with an inner coupling member of the deployment shaft attached and showing an exemplary spring-biased finger, and FIG. 28B shows deployment of the spring-biased finger to fold the elongated cinching element against the junction housing once the inner coupling member is removed;

FIG. 29 is a side elevational view of an alternative configuration for folding the elongated cinching element through the use of two spring-biased fingers;

FIG. 30A is a schematic view of an adjustable annuloplasty ring of the present application having a cinching element and junction housing that permit but limit expansion of the ring, and FIG. 30B is a view of the annuloplasty ring after expansion showing the cinching element limiting further expansion;

FIG. 31A is a schematic view of another adjustable annuloplasty ring having a cinching element and a secondary cord within the ring to limit expansion thereof, and FIG. 31B shows the annuloplasty ring after expansion with the cinching element broken and the cord expanded taut;

FIG. 32 is a perspective view of an alternative junction housing for an adjustable annuloplasty ring;

FIG. 33 is an exploded perspective view of the alternative junction housing of FIG. 32;

FIG. 34 is an assembled perspective view of the alternative junction housing, and FIG. 34A is a radial sectional view therethrough taken along line 34a-34a;

FIG. 35 is a circumferential sectional view through the alternative junction housing of FIG. 32 and a portion of the connected annuloplasty ring;

FIG. 36 is a schematic view of an alternative cinching element utilizing tension sutures connected to a metallic wire;

FIG. 37A is a perspective view of a junction housing of an alternative annuloplasty ring having a suture loop for a cinching element and shown a portion of a modified deployment system, and FIG. 37B is a perspective view of the junction housing after cinching and severing the suture loop;

FIG. 38 is a schematic view of an alternative annuloplasty ring utilizing a suture loop for a cinching element and two pairs of tension suture loops attached to opposite ends of the cinching element;

FIG. 39 is a perspective view of a junction housing for the annuloplasty ring of FIG. 38 after cinching and removal of the tension suture loops;

FIG. 40 is a perspective view of another saddle-shaped support body for an annuloplasty ring formed by laser cutting a nitinol tube;

FIG. 41 is a perspective view of a linear nitinol tube after having been laser cut, and FIG. 42 is a different perspective view of the support body of FIG. 40 after the linear nitinol tube has been shape set; and

FIG. 43A is a perspective view of a lower portion of a shape setting mandrel having the laser cut support body positioned thereon, and FIG. 43B is a mandrel assembly that sandwiches the laser cut support body for shape setting.

### DETAILED DESCRIPTION

The right ventricle and left ventricle are separated from the right atrium and left atrium, respectively, by the tricuspid valve and mitral valve; e.g., the atrioventricular valves. Though correction of the mitral annulus is the primary focus of the present application, it should be understood that certain characteristics of the annuloplasty rings described herein may equally be used to treat the tricuspid valve, and thus the claims should not be constrained to the mitral ring unless expressly limited.

The term “axis” in reference to the illustrated annuloplasty rings, and other non-circular or non-planar rings, refers to a line generally through the centroid of the ring periphery when viewed in plan view. “Axial” or the direction of the “axis” can also be viewed as being parallel to the average direction of blood flow within the valve orifice and thus within the ring when implanted therein. Stated another way, an implanted mitral ring orients about a central flow axis aligned along an average direction of blood flow through the mitral annulus from the left atrium to the left ventricle. The plan views of the annuloplasty rings illustrated herein are as looking from the atrial side in the direction of blood flow. For the purpose of orientation, therefore, the atrial side of the ring is up and the ventricular side is down.

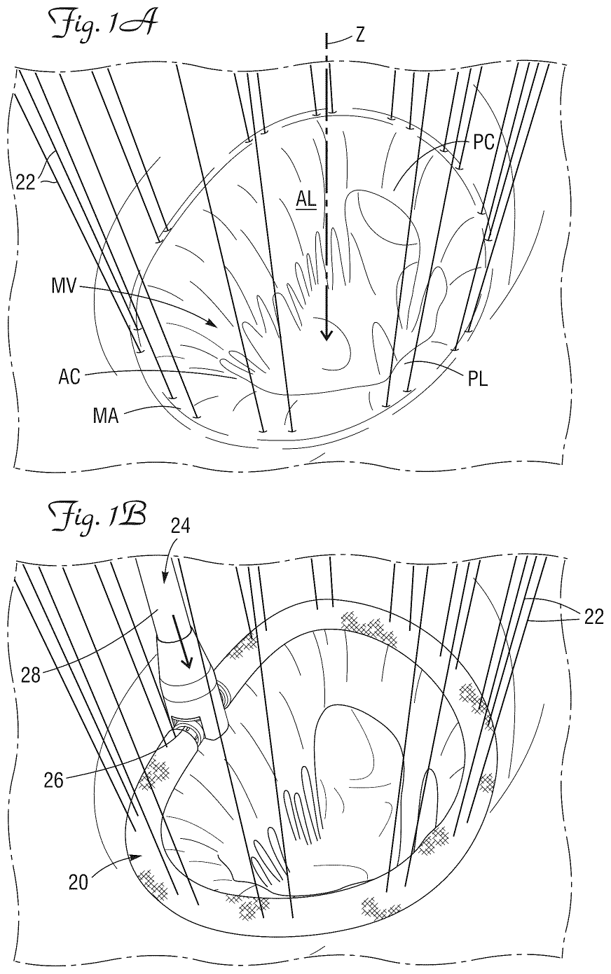

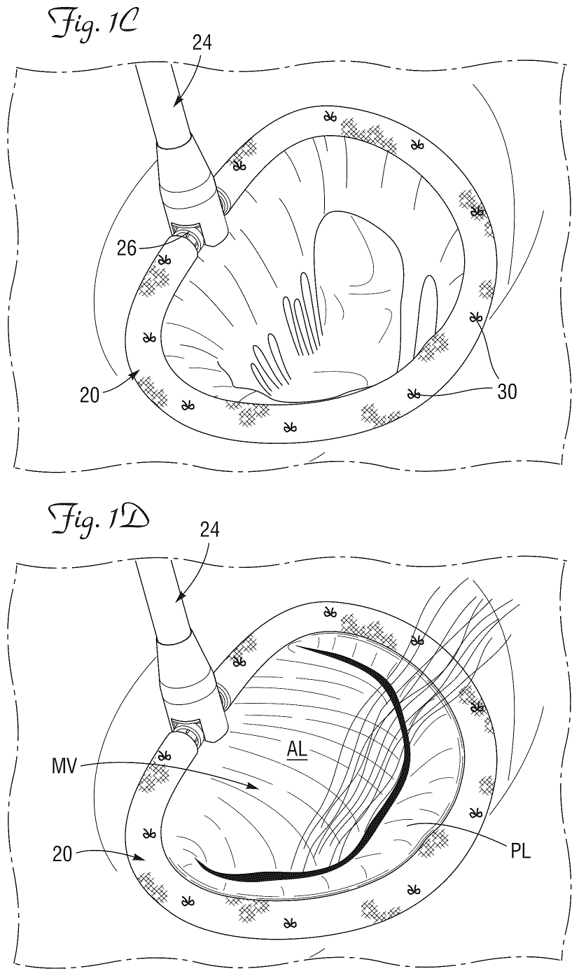

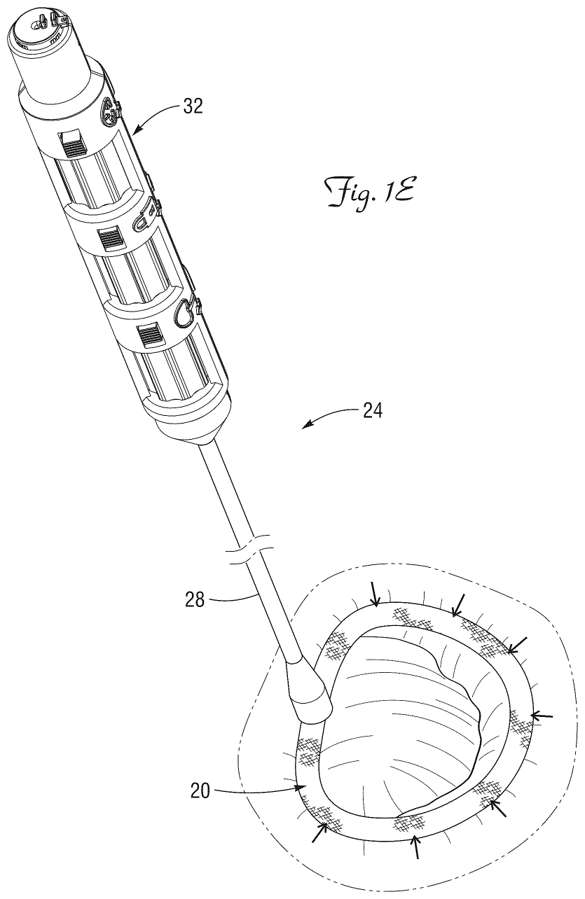

FIG. 1A is a schematic perspective view from the atrial side of a mitral valve MV with posterior being down and anterior being up. The mitral valve MV primarily comprises a pair of floppy coapting leaflets—an anterior leaflet AL and a posterior leaflet PL—secured around their outer edges to a fibrous mitral annulus MA. The anterior leaflet AL attaches to a somewhat straighter anterior fibrous portion of the mitral annulus MA, which makes up about one-third of the total mitral annulus circumference. The anterior fibrous annulus forms part of the central fibrous skeleton of the heart, and the two ends are called the fibrous left and right trigones. The arcuate muscular portion of the mitral annulus MA constitutes the remaining two-thirds of the mitral annulus, and the posterior leaflet PL attaches thereto. An anterior commissure AC and a posterior commissure PC at the junction of the two leaflets on each side are located just posterior to each fibrous trigone.

The peripheral mitral annulus MA is often described as kidney bean or D-shaped with a somewhat straighter side adjacent the anterior leaflet AL and a more rounded or convex side adjacent the posterior leaflet PL. The mitral annulus MA is typically viewed as having a major axis across its widest part, approximately between the commissures AC and PC, and a perpendicular minor axis that generally bisects both the anterior leaflet AL and posterior leaflet PL. A central axis Z flow direction is arbitrarily defined at the intersection of the major and minor axes.

FIG. 1A-1E illustrate several steps of implanting and adjusting an annuloplasty ring 20 of the present application. The procedure for gaining access to the mitral annulus involves making an incision through the patient's sternum (sternotomy) and then stopping the heart and rerouting blood flow through a heart-lung “cardiopulmonary” bypass machine. The mitral annulus is exposed through the left atrium. At this point a measurement is taken of the mitral annulus MA, typically by measuring the width across the major axis between the anterior and posterior commissures AC, PC as well as the leaflet area. Annuloplasty rings are conventionally provided in sizes between 24 and 40 mm, in 2 mm increments. The final size of the adjustable annuloplasty ring 20 as described herein is not constrained to these 2 mm increments, but is provided in a number of sizes per usual to best match the particular mitral annulus being repaired.

Once the mitral annulus MA is accessible, as seen in FIG. 1A, a series of anchoring sutures 22 are pre-installed around the annulus. The anchoring sutures 22 are each looped through the atrial side to the ventricular side of the annulus, and then passed back up so that there are multiple pairs of sutures coming up out of the implantation site. In the illustrated embodiment, there are twelve pairs of sutures shown, though more or less may be utilized. Pledgets or small strips of fabric or other such backing (not shown) may be installed on the ventricular side of the suture loops to help prevent suture pull-through.

FIG. 1B shows an annuloplasty ring 20 of the present application being advanced down to the mitral annulus MA along the pairs of sutures 22. A distal end of a delivery or deployment system 24 attaches to a junction housing 26 at a midpoint of an anterior segment of the annuloplasty ring 22 and is used to parachute the ring down the array of sutures 22. The deployment system 24 has a shaft 28 long enough to extend from outside the body to the surgical site, the shaft being connected to a proximal control handle as will be described below.

FIG. 1C shows the implanted annuloplasty ring 20 after the pairs of sutures 20 have been tied off into knots 30 and severed close to the ring. The distal end of the deployment system 24 remains attached to the junction housing 26. Following the process of anchoring the annuloplasty ring 20 to the mitral annulus MA, the left atrium is closed around the deployment system 24 with one or more purse string sutures. All other incisions are closed to prevent blood loss, with the deployment system 24 remaining such that it may be controlled by its proximal control handle outside the body. Subsequently, the patient is weaned off of cardiopulmonary bypass and heart restarted.

Adjustment of the annuloplasty ring 20 to optimize the repair is guided by a visualization technique such as transesophageal echocardiography (TEE) or in rare cases fluoroscopy, mainly focusing on residual mitral regurgitation, degree of leaflet coaptation and the presence of transmitral gradients. For example, FIG. 1D illustrates the mitral valve MV during systole when the anterior leaflet AL and posterior leaflet PL come together or coapt. In this illustration, mitral regurgitation is indicated by the escaping blood flow which can be seen on TEE. This means that the leaflets AL, PL are not coapting, which may be corrected by reducing the size of the annuloplasty ring 20. Even if there is no regurgitation seen, the surgeon may still decide to adjust the ring size in order to obtain a larger surface of coaptation.

FIG. 1E shows the annuloplasty ring 20 being reduced in size using the proximal control handle 32, which again is outside the body. Cinching the annuloplasty ring 20 in this regard brings the leaflets AL, PL closer together, and also the AL-PM diameter when desired, thus improving coaptation. In the illustration, the mitral valve MV is closed during systole with the leaflets coapting and no regurgitation detected. The annuloplasty ring 20 may be reduced in size by at least 2 mm across the major axis, equivalent to one standard ring size. Further reduction in major axis dimension may be provided up to 4 mm. Following ring size adjustments, the deployment system 24 is disengaged from the annuloplasty ring 20 and removed from the body. The purse string suture through the left atrial wall and any other openings are then closed to complete the procedure.

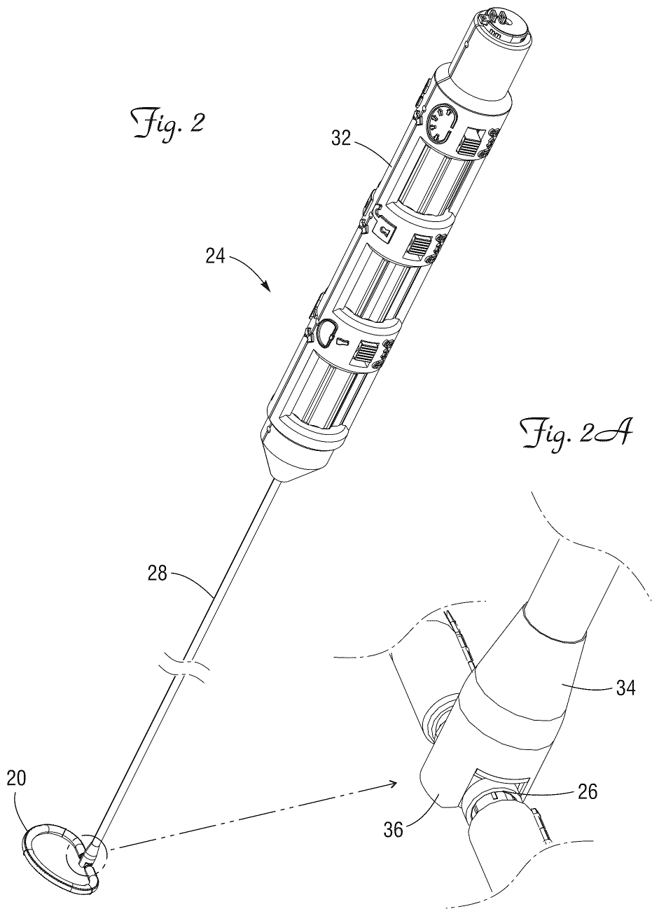

FIG. 2 is a perspective view of an exemplary deployment system 24 for the adjustable annuloplasty ring 20, and FIG. 2A is an enlarged view of the distal end of a deployment shaft 28 coupled to the junction housing 26 of the annuloplasty ring. The proximal control handle 32 is shown in approximately the actual proportional size relative to the annuloplasty ring 20, and generally comprises a cylindrical body having a plurality of controls thereon. The elongated shaft 28 is shown with a break, but may have a length of between 40-80 cm. The shaft is configured to have some lateral flexibility, but is stiff under compression and torque.

The distal end of the shaft 28 terminates in a tapered shroud or housing 34 with a pair of diametrically-opposed extensions 36 that straddle the junction housing 26 on radially inner and outer sides. Another way to see the housing 34 is that the extensions 36 define cutouts therebetween aligned around the periphery of the annuloplasty ring 20. As will be explained below, the distal housing 34 encloses a number of operative elements which engage the junction housing 26 to adjust the size of the annuloplasty ring 20 and perform several other tasks.

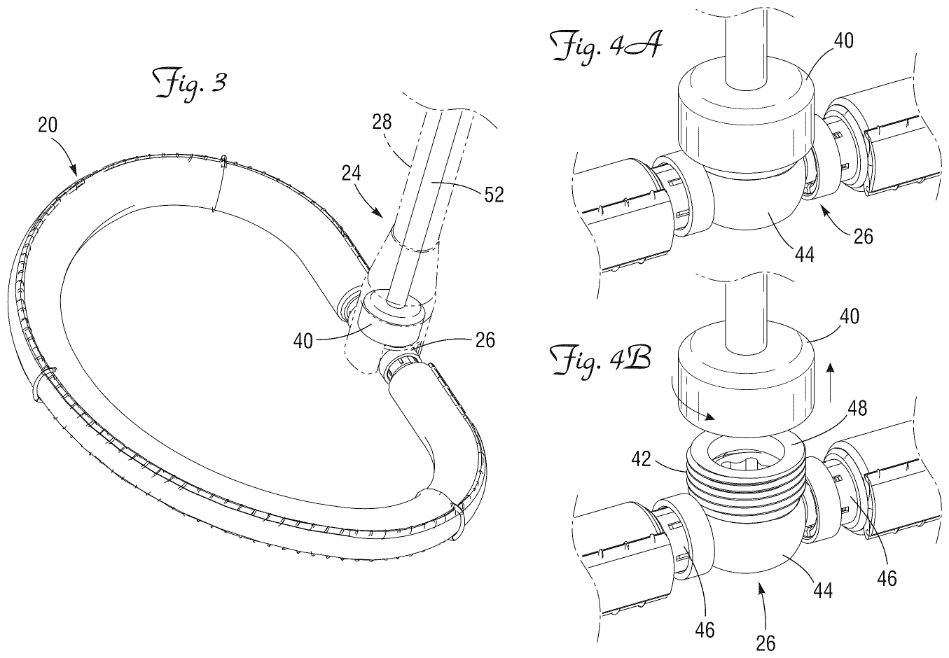

FIG. 3 is a perspective view of the distal end of the deployment shaft 28 shown in phantom engaged with the junction housing 26, while FIG. 4A illustrates an inner coupling member 40 of the deployment system 24 engaged with the junction housing. One way to couple the deployment system 24 with the annuloplasty ring is by using an internally threaded coupling member 40 which engages external threads 42 on the junction housing 26. The junction housing 26 has a main body 44 defining a three-way connection. Oppositely-directed side legs 46 extend along the periphery of the annuloplasty ring 20 and connect with an internal passage therein. A single upper port 48 having the external threads 42 thereon projects upward perpendicularly relative to the side legs 46. The deployment shaft 28 connects to the annuloplasty ring 20 in line with the upper port 48, and the coupling member 40 screws onto the external threads 42. FIG. 4B shows disengagement (unscrewing and retraction) of the coupling member 40 from the junction housing 26, which is how the deployment system 24 is decoupled from the annuloplasty ring 20.

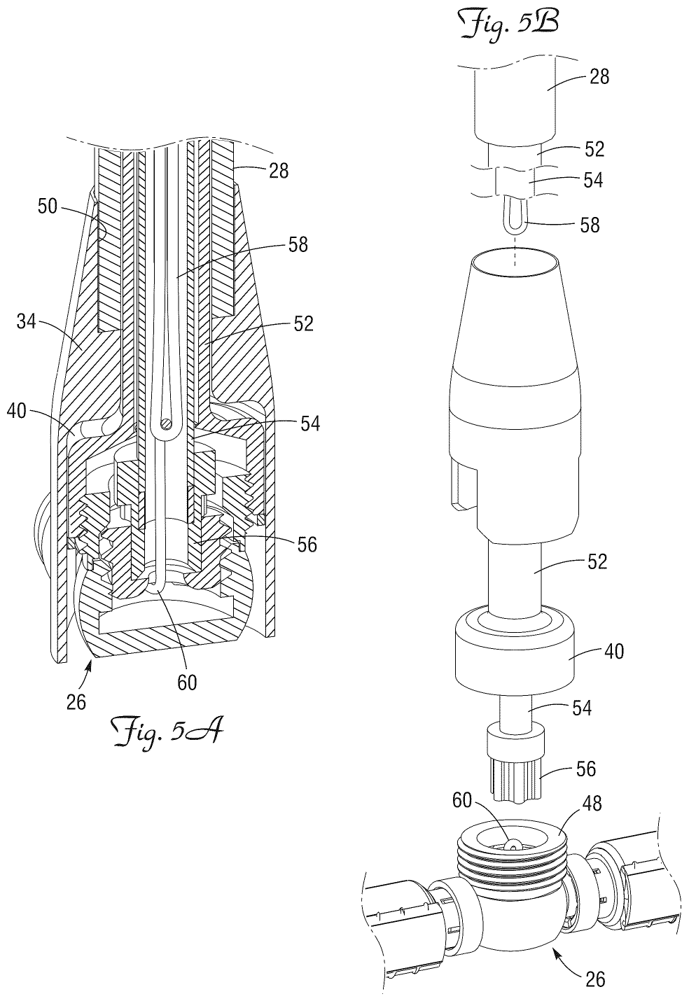

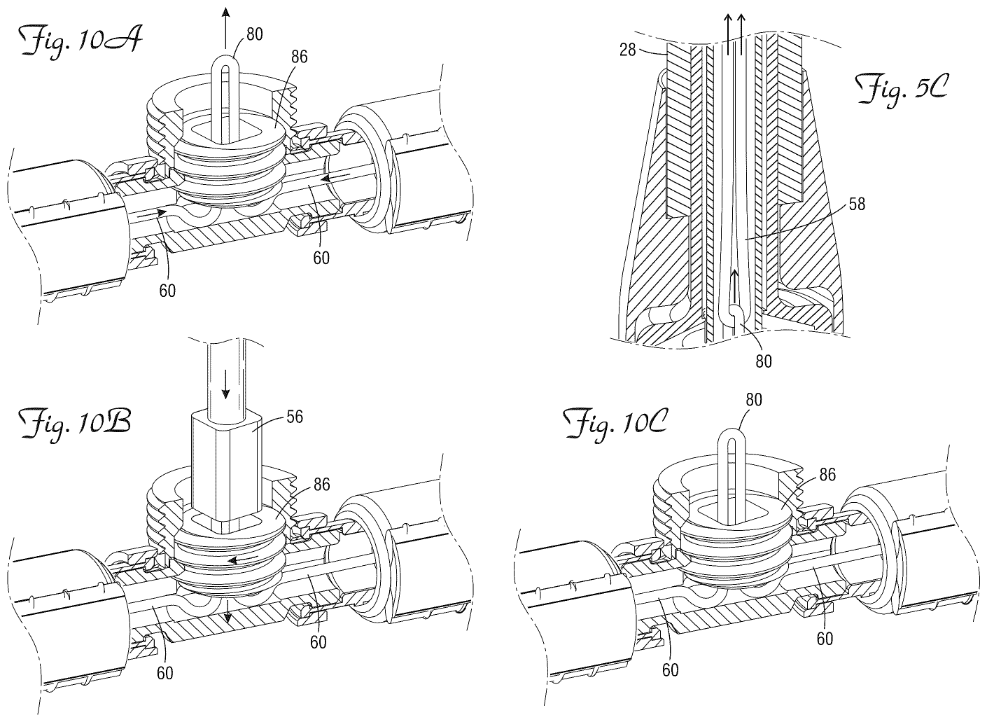

FIG. 5A is a vertical sectional view of the interaction between the distal end of the deployment shaft 28 and the junction housing 26, while FIG. 5B is an exploded perspective view of the components thereof. The outer shroud or housing 34 has an upper recess 50 into which the distal end of the deployment shaft 28 is fixed, and a wider internal distal end which matches the cup-shape of the coupling member 40. The coupling member 40, in turn, is located at the distal end of an elongated tubular first shaft 52 which extends through a lumen of the deployment shaft 28 to the control handle 32. A tubular second shaft 54 slides and rotates concentrically within the first shaft 52 and has a distal non-circular torque driver 56 thereon. As will be explained below, the torque driver 56 engages an element within the junction housing 26 to lock the shape of the adjustable annuloplasty ring 20.

A final element of the deployment system 24 is an adjustment cable 58 that extends from the control handle 32 through the lumen of the tubular second shaft 54 to the junction housing 26. FIG. 5A shows the adjustment cable 58 in the form of a loop which extends through a subloop of the cinching element 60 that projects up through a central opening in the junction housing 26. FIG. 5C (sheet 12) is a vertical sectional view showing tension being applied by the adjustment cable 58 through the deployment shaft 28 to the cinching element 60. In one embodiment, the adjustment cable 58 comprises a filament, such as a polymeric suture or metal cable, while the cinching element 60 is formed by a flexible metallic wire. The cinching element 60 may be formed of nitinol, stainless steel, or a cobalt chromium (CoCr) alloy, and will be described in greater detail below.

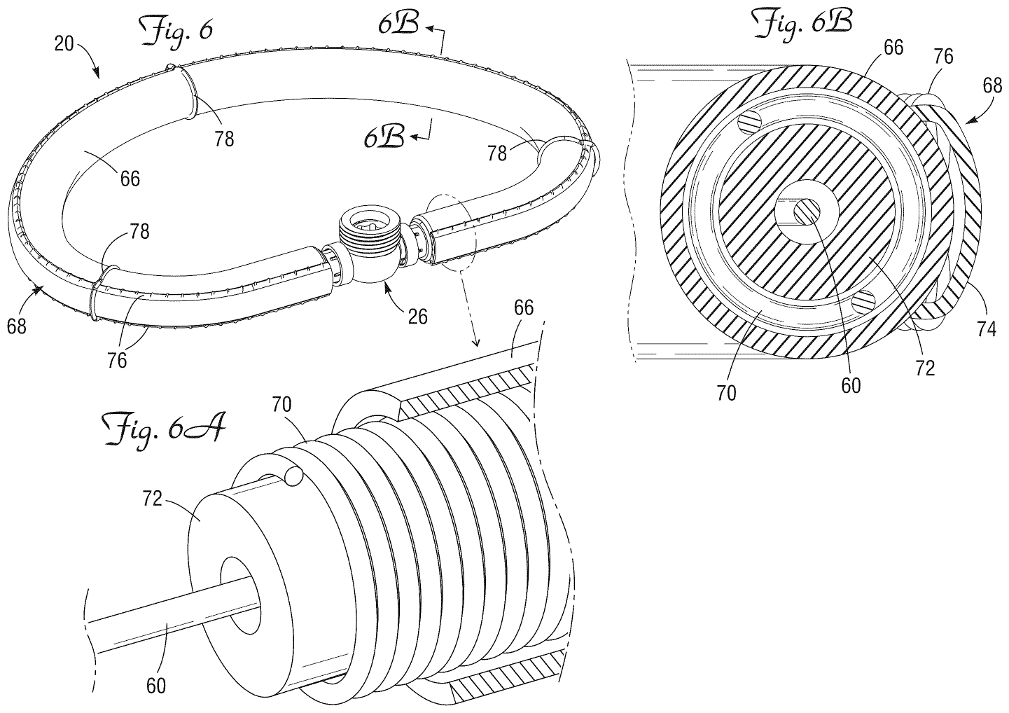

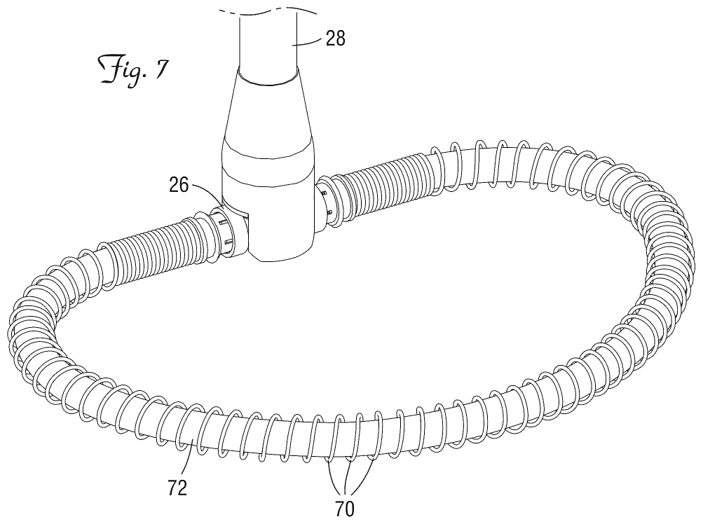

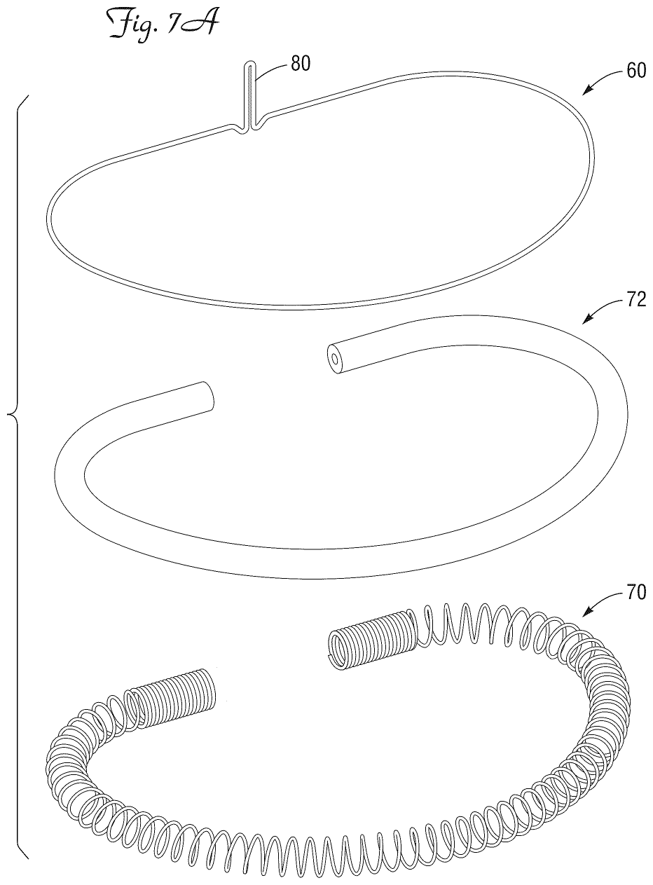

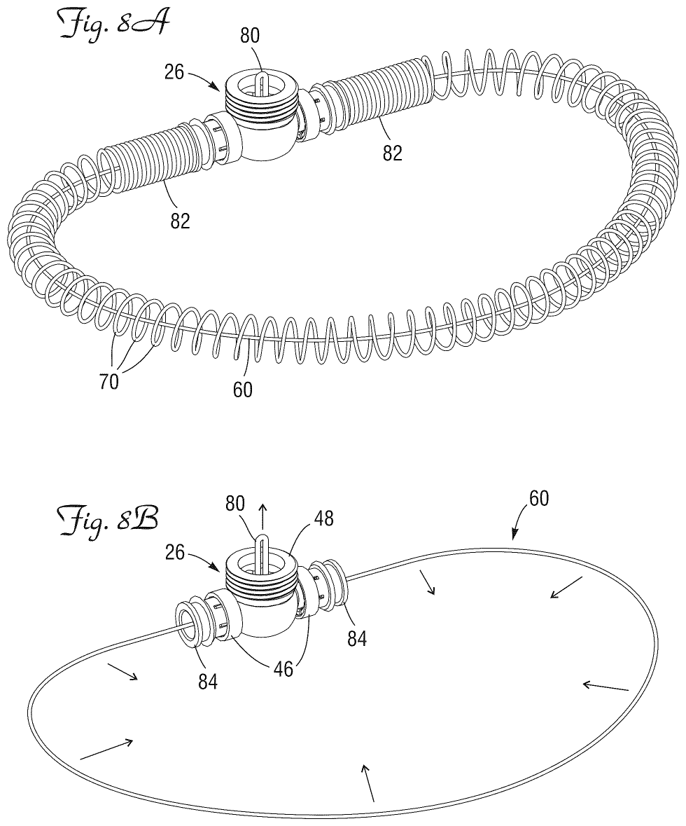

FIG. 6 is a perspective view of the exemplary adjustable annuloplasty ring 20, and to better explain the construction thereof, FIG. 6A is an enlarged cutaway view of one portion and FIG. 6B is a radial sectional view thereof. As mentioned above, the annuloplasty ring defines a rounded D-shape with a straighter anterior segment 62 in which is centered the junction housing 26 opposite a rounded posterior segment 64. Aside from the junction housing 26, the exterior of the annuloplasty ring 20 is covered with a tubular fabric cover 66 with a sewing cuff 68 added to a peripheral outer edge. The fabric cover 66 closely surrounds a coiled body 70 through which a hollow filler member 72 extends. Finally, the cinching element 60 passes around the entire periphery of the annuloplasty ring 20 within a central lumen in the filler member 72. The filler member 72 acts as a cushion between the cinching element 60 and coiled body 70.

The radial sectional view of FIG. 6B shows the extent that one embodiment of a sewing cuff 68 extends outward from the fabric cover 66, which will be more fully discussed below. The sewing cuff 68 comprises a ring of fabric 74 formed by a single layer that is attached by a series of stitches 76 along the upper and lower edges to the fabric cover 66. In one embodiment, the stitches 76 are colored differently than both the fabric cover 66 and ring of fabric 74 so that the upper and lower edges of the ring of fabric is accentuated. This serves to inform a surgeon where to pass the anchoring sutures—e.g., between the stitches 76 along the upper and lower edges of the ring of fabric 74. The outer cover 66 further has a plurality of discrete marker bands 78 sewn thereto, at positions indicating the points on the ring corresponding to the fibrous trigones and midpoint of the posterior segment of the mitral annulus. The marker bands 78 help position the ring 20 around the annulus during implantation.

To further explain the structure of the annuloplasty ring 20, FIG. 7 is a perspective view of the distal end of the deployment shaft 28 coupled to the ring having the outer cover removed, and FIG. 7A is an exploded perspective view of the inner components of the annuloplasty ring, while FIG. 8A is a perspective view of the ring with the outer cover 66 and inner filler member 72 removed, and FIG. 8B is the same perspective view with the coiled body 70 removed illustrating the cinching element 60. As can be seen from these images, each of the components of the cinching element 60, coiled body 70 and filler member 72 approximately define the rounded D-shape of the annuloplasty ring 20, and are preferably pre-formed into such a shape. For example, the coiled body 70 is desirably a highly elastic metal such as nitinol and may be heat set into the illustrated shape.

As seen best in FIG. 7A, both the coiled body 70 and filler member 72 define two free ends on their straighter anterior sides that are spaced apart to accommodate the junction housing 26 therebetween. In contrast, the cinching element 60 forms a continuous loop that passes into the interior of the junction housing 26 and projects upward in a vertical subloop 80 through the upper port 48 thereof (FIG. 8A). As seen by the arrows in FIG. 8B, pulling on the subloop 80 constricts the remainder of the cinching element 60 to reduce the circumference of the annuloplasty ring 20. That is, both the coiled body 70 and filler member 72, not to mention the outer fabric cover 66, are flexible in terms of constriction, and thus the cinching element 60 determines their final size. As will be seen, the cinching element 60 is locked in its reduced diameter constricted state within the junction housing 26. Due to the metallic nature of the cinching element 60 the ring size remains the same after constriction and is not subject to stretching from material creep.

The cinching element 60 provides an advantage over prior adjustable annuloplasty rings in that it remains in a continuous loop before and after ring adjustment. Prior annuloplasty rings having sutures that extend around the periphery of the annuloplasty ring required a tool to cut them, adding complexity to the deployment system. Furthermore, once cut, the free ends of the sutures sometimes presented sharp trimmed ends extending away from the annuloplasty ring potentially leading to tissue irritation or worse. Finally, sutures have been exclusively used in the past which are made of a polymer and subject to material creep, thus allowing the ring to expand over time. The cinching element 60, on the other hand, is made of a metal such as a cobalt chromium alloy which resists creep.

FIGS. 8A-8B also indicate how the junction housing 26 connects to the body of the annuloplasty ring 20. The coiled body 70 in the illustrated embodiment has a series of regularly spaced apart coils around most of its periphery except at its free ends 82 which are more tightly coiled, forming something of a tube. The tubular free ends 82 abut outer flanges formed on the side legs 46 of the junction housing 26. Although not shown, the outer cover 66 extends inward over the free ends 82 and is secured within a pair of grooves 84 defined between flanges on the side legs 46. The outer cover 66 may have smaller diameter ends formed by a cinched portion or constriction ring or the like to hold the ends within the grooves 84. The filler body 72 may terminate co-extensively with the coiled body 70, or may extend a short distance into the hollow side legs 46.

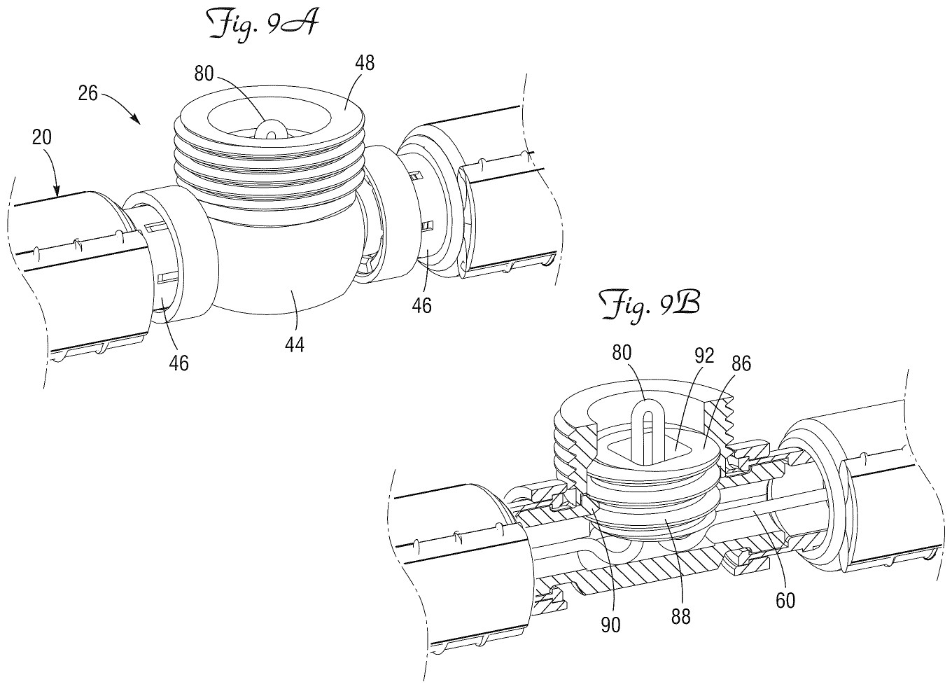

FIG. 9A is an enlarged perspective view of the junction housing 26 of the adjustable annuloplasty ring 20, and FIG. 9B is a cutaway view showing internal components thereof. As mentioned above, the junction housing 26 has a three-way connection with a main body 44 and oppositely-directed side legs 46. The upper port 48 defines an upper opening of a vertical channel which leads down to a horizontal channel through the side legs 46. As mentioned, the cinching element 60 extends around the ring and into the channels of the side legs 46, and then upward in the subloop 80 so as to be accessible through the upper port 48. The subloop 80 passes through the hollow center of a locking screw 86 positioned in the vertical channel. The locking screw 86 has external threads 88 which engage internal threads defined within the vertical channel of the junction housing 26. For example, FIG. 9B illustrates an inward projection 90 formed by the side legs 46 that may be shaped to mate with the external threads 88. The locking screw 86 has a non-circular internal throughbore 92 to enable rotation by the torque driver 56 (See FIG. 5B). In the embodiment illustrated in FIGS. 9-10, the through bore 92 has a square configuration, though the torque driver 56 is shown with an alternative star-shaped configuration. Either can be utilized, and both are shown for just that purpose.

Now with reference to FIGS. 10A-10C and 5C, the process for locking the cinching element 60 is explained. To recap, however, cinching the annuloplasty ring 20 and locking the cinching element 60 is done after anchoring the annuloplasty ring to the mitral annulus, as was described with reference to FIGS. 1A-1E. That is, the annuloplasty ring 20 has been anchored to the annulus with sutures, and then in FIG. 1E the ring is constricted via a mechanism within the control handle 32 to reduce the size of the annulus. Furthermore, this procedure is done under visualization after closing up the access incisions and restarting the patient's heart so as to observe any regurgitation through the mitral valve. The deployment shaft 28 extends from outside the patient's body into engagement with the annuloplasty ring 20 during this process. Prior to cinching the annuloplasty ring 20, the surgeon observes the mitral valve function and if regurgitation is present, incrementally reduces the size of the ring 20 and then locks it when there is no more regurgitation.

FIG. 5C illustrates the loop of the adjustment cable 58 being pulled in a proximal direction from the control handle 32. The adjustment cable 58 passes through the subloop 80 of the cinching element 60, and thus pulls the subloop up through the hollow locking screw 86 and reduces the circumference of the cinching element around the annuloplasty ring 20, as was depicted in FIG. 8C.

Once the proper size of the annuloplasty ring 20 is determined, the locking screw 86 is advanced by rotating and advancing the torque driver 56, as seen in FIG. 10B. Eventually, the lower end of the locking screw 86 pinches the diverging strands of the cinching element 60 against the interior lower wall of the junction housing 26. The optimal extent to which the locking screw 86 advances and clamps against the cinching element 60 within the junction housing 26 may be determined empirically, and then implemented in a number of ways. For example, the locking screw 86 may be calibrated in terms of its thread pitch to advance a particular axial distance upon a predetermined angular rotation, monitored at the control handle 32. Alternatively, the torque driver 56 may be actuated within the control handle 32 using a clutch mechanism which slips upon a predetermined reaction torque being sensed. Still further, the female threads within the junction housing 26 may be configured like a standard locking nut with a nylon insert or the like which provides a frictional resistance to further advancement of the locking screw 86 at a particular point. A nylon insert provides a further advantage in preventing reverse rotation of the locking screw 86.

Finally, after advancing the locking screw 86 to firmly clamp against the cinching element 60, as seen in FIG. 10C, the deployment system 24 is disengaged from the junction housing 26, such as was shown and described with respect to FIGS. 4A-4B, and withdrawn from within the body, while closing up any remaining incisions. This is done simply by severing or releasing one strand of the looped adjustment cable 58 from the control handle 32, and then pulling the released strand from within the subloop 80. No special cutter or other such instrument at the distal end of the deployment system 24 is required to sever the adjustment cable 58 or the cinching element 60. What is left is the subloop 80 projecting somewhat from the upper port 48 of the junction housing 26. Because of the rounded nature of the subloop 80, it does not present any danger of lacerating or otherwise irritating tissue or leaflets within the mitral valve.

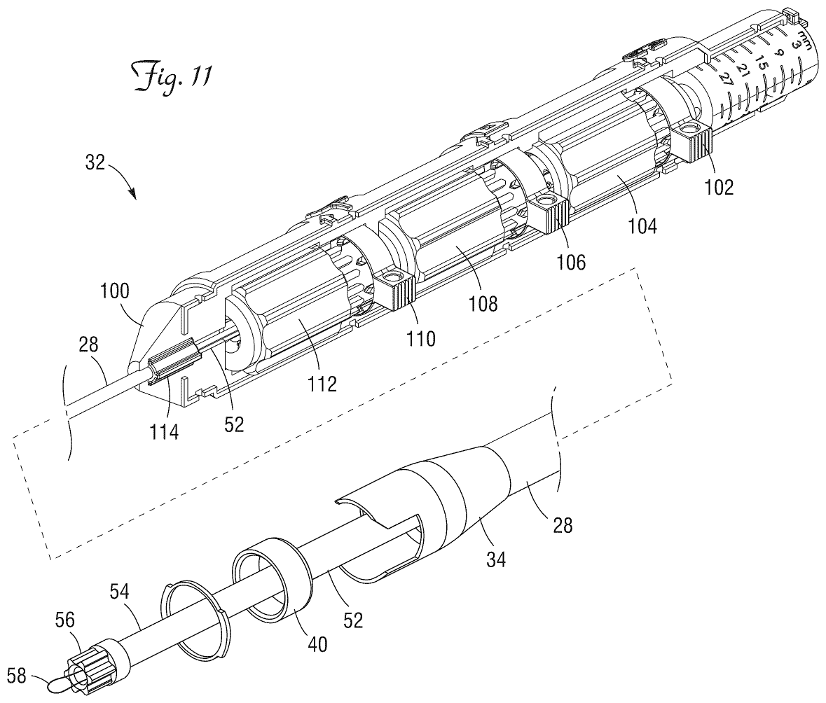

Exemplary aspects of the proximal control handle 32 will now be described with respect to FIGS. 11, 12 and 12A. The control handle 32 features a wand-like outer housing 100 which is generally cylindrical in exterior profile and sized to be easily manipulated with one hand by a surgeon or technician. The housing 100 may be formed by a pair of similar housing halves which are secured together with fasteners, adhesives, or the like. As seen in FIG. 12, the housing 100 defines a series of internal chambers which receive rotatable actuator wheels and corresponding locking collars. More particularly, and with reference again to FIG. 11 and moving from a proximal end to a distal end, the control handle 32 has a first locking collar 102 and actuator wheel 104 for cinching the annuloplasty ring 20, a second locking collar 106 and actuator wheel 108 for fixing the size of the annuloplasty ring, and a third locking collar 110 and actuator wheel 112 for disengaging the deployment system 24 from the annuloplasty ring. Each of the actuator wheels 104, 108, 112 rotates about a central axis of the control handle 32, and each of the locking collars 102, 106, 110 slides axially between two positions on the control handle.

Each of the actuator wheels 104, 108, 112 has a tubular configuration with a central throughbore so that shafts, wires, etc. can pass axially therethrough. Each of the wheels 104, 108, 112 further has a scalloped or otherwise uneven outer surface to facilitate rotation, especially in the sometimes wet environment of the surgery suite. The uneven outer surface of each of the wheels 104, 108, 112 projects through a series of rectangular windows 101 formed in the outer housing 100 to enable the user to easily rotate the wheels. In the illustrated embodiment, the windows 101 are provided on opposite sides of the housing 100 so that a user can easily embrace the control handle 32 with the palm of one hand while applying torque to anyone of the wheels 104, 108, 112 with the thumb and finger.

FIG. 12 shows that each of the wheels 104, 108, 112 has a proximal extension 103 around which a ring-like body of the locking collars 102, 106, 110 is arranged to slide axially. Each of the locking collars 102, 106, 110 slide axially along the associated proximal extension 103 and permits rotation of the respective actuator wheel 104, 108, 112 when in the rightmost position. Each of the locking collars 102, 106, 110 has a pair of oppositely-directed tabs 105 that project outward through axial slots 107 in the housing 100, the axial slots determining the extent of movement of each of the locking collars. The locking collars 102, 106, 110 each has a series of circumferentially-spaced internal splines 109 that mesh with a series of circumferentially-spaced axial ribs 11 on the respective actuator wheel 104, 108, 112. When each locking collar 102, 106, 110 is in its left position, with the tabs 105 to the left within the slots 107, the splines 109 engage with the ribs 111 to prevent the respective actuator wheel 104, 108, 112 from rotating.

With reference back to FIG. 11, an enlargement of the components at the distal end of the deployment system 24 are shown. In particular, the outer deployment shaft 28 and the distal housing 34 are shown. The deployment shaft 28 enters the distal end of the control handle housing 100, and is fixed rotationally therein by a non-circular end sleeve 114 held within a similarly-shaped recess in the handle. The coupling member 40 and first shaft 52 are shown extending distally from the housing 34, and the tubular second shaft 54 and torque driver 56 from the first shaft. The first and second shafts 52, 54 are rotatable within the outer shaft 28, with the second shaft being linearly movable therein. Finally, the tension adjustment cable 58 is seen projecting from within the lumen of the second shaft 54.

Now referring to FIG. 12, the first shaft 52 is seen extending as far as to within the distalmost actuator wheel 112. Although not shown, the first shaft 52 is fixed with respect to an internal bore of the actuator wheel 112 which is thus capable of rotating the first shaft. The second shaft 54 continues in a proximal direction and terminates at a non-circular head 113 that meshes with and is rotated by a similarly shaped inner bore of a clutch member 115. The clutch member 115 has a cylindrical exterior which fits within an inner chamber of the middle actuator wheel 108. Rotation of the middle actuator wheel 108 rotates the clutch member 115 until a predetermined reaction torque is met. The non-circular head 113 of the second shaft 54 is biased in a distal direction by a spring 117 fitted over the distal end of a threaded rod 119. The threaded rod 119 in turn engages internal threads within the middle actuator wheel 108. The distal bias of spring 117 maintains a distal force on the second shaft 54 so that the torque driver 56 at its distal end stays within the throughbore 92 of the locking screw 86 (see FIG. 10B).

The tension adjustment cable 58 extends proximally through the second shaft 54 and, as seen in FIG. 12, through the clutch member 115, middle actuator wheel 108, through the proximal actuator wheel 104, and is secured at both free ends to a tension adjuster 116. FIG. 12A is an enlarged view of the connection between the tension adjustment cable 58 and control handle 32. The tension adjuster 116 has a distal stub 117 with a lateral hole to which one free end of the tension adjustment cable 58 attaches, such as with a loop and crimp as shown. The other free end of the tension adjustment cable 58 passes all the way through the tension adjuster 116 and is secured to a cleat and cutting guide assembly 118 in the form of small tabs with holes therethrough. The tension adjuster 116 further includes a ring reduction indicator 120 on a distal end. External threading 121 on the distal end of the tension adjuster 116 mesh with internal threads 122 on the first actuator wheel 104. Rotation of the first actuator wheel 104 causes axial displacement of the tension adjuster 116. Rotation in one direction displaces the tension adjuster 116 proximally so as to pull the tension adjustment cable 58 and constricted the annuloplasty ring. As the tension adjuster 116 moves proximally (to the right), the ring reduction indicator 120 gradually projects farther out of the proximal end of the control handle housing 100. The indicator 120 has a series of millimeter markings thereon which are calibrated to correspond to reduction in the major diameter of the annuloplasty ring.

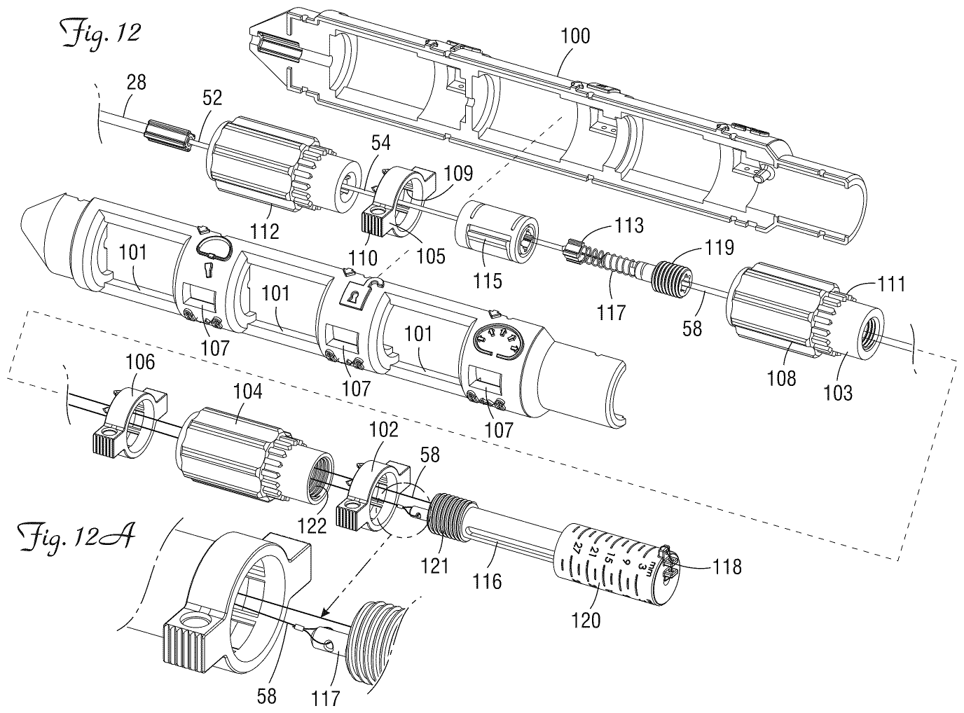

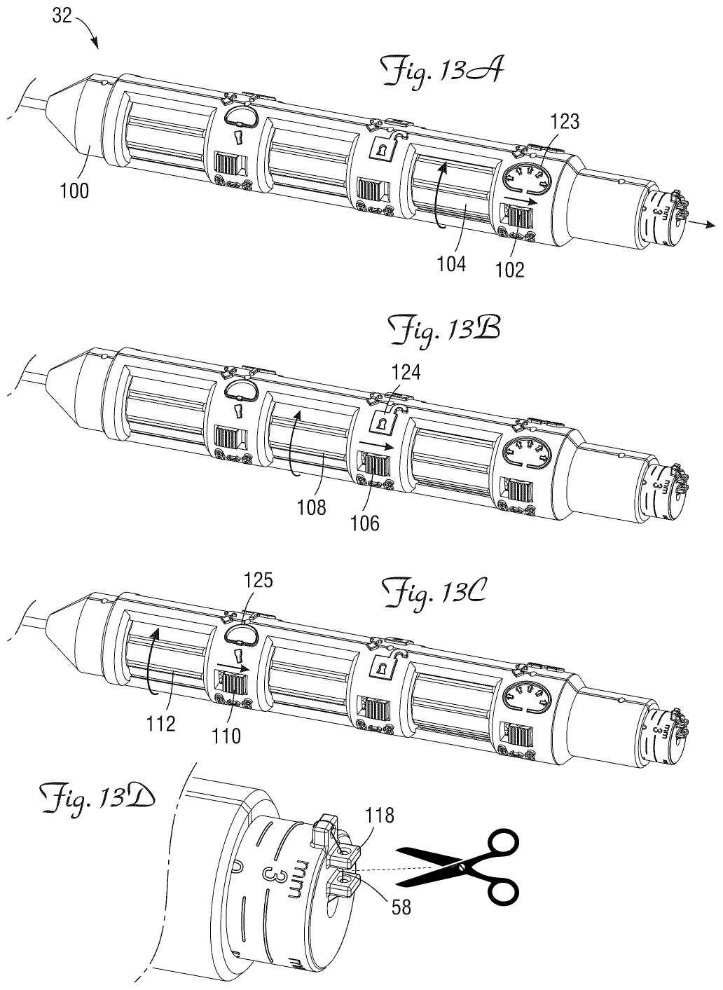

FIG. 13A-13C are perspective views of the proximal control handle 32 during operation thereof, and FIG. 13D is an enlarged view of an operation to sever the adjustment cable 58 within the deployment system. First, FIG. 13A illustrates rightward displacement of the first locking collar 102 which releases the first actuator wheel 104 for rotation. As suggested by a first graphic indicator 123, rotation of the first actuator wheel 104 adjusts the size of the annuloplasty ring 20. More particularly, rotating the first actuator wheel 104 displaces the tension adjuster 116 (to the right) to reduce the size of the annuloplasty ring 20. The indicator 120 projects out of the housing 100 and tracks the incremental decrease in ring size in mm of major axis reduction. As mentioned above, the surgeon monitors blood flow through the mitral valve looking for excessive regurgitation. At some magnitude of ring constriction, regurgitation will be minimized.

FIG. 13B shows movement of the middle locking collar 106 to the right which frees up the middle actuator wheel 108 to rotate. As suggested by a second graphic indicator 124, rotation of the middle actuator wheel 108 locks the desired size of the annuloplasty ring 20. As the actuator wheel 108 rotates, the clutch member 115 also rotates to, in turn, rotate the non-circular head 113 of the second shaft 54. The second shaft 54 rotates the torque driver 56 at its distal end to tighten the locking screw 86 and clamp against the cinching element 60 (see FIG. 10B). At a predetermined reaction torque, the clutch member 115 slips to stop rotation of the torque driver 56. The slippage will ideally be felt or heard as audible clicking to notify the user. The predetermined torque applied to the locking screw 86 is calibrated to reliably clamp the cinching element 60 in a cinched position with a desired annulus reduction of the ring 20.

Subsequently, the adjustment cable 58 is decoupled from the subloop 80 of the cinching element 60. FIG. 13D shows a schematic cutter or scissors being used to sever the portion of one of the lengths of the adjustment cable 58 between tabs of the cleat and cutting guide assembly 118. This frees up the adjustment cable 58 to be pulled free of the subloop 80 when the deployment system 24 is removed from the body.

Finally, FIG. 13C shows rightward displacement of the third locking collar 110 which releases the distal actuator wheel 112 for rotation. As suggested by a third graphic indicator 125, rotation of the distal actuator wheel 112 decouples the deployment system 24 from the annuloplasty ring 20. Namely, the actuator wheel 112 rotates the first shaft 52 and distal coupling member 40 of the deployment system 24 (see FIG. 4B). Once the coupling member 40 detaches from the threading on the junction housing 26, the deployment system 24 is ready to be removed from the body.

As mentioned above, the sewing cuff 68 is added to increase the radial extent of the material used to anchor the ring. FIG. 14 is a schematic view of an anterior segment of an adjustable annuloplasty ring 130 without the sewing cuff 68 and having a deployment shaft 132 attached thereto. An outer cover 134 is partially removed to illustrate the tightly coiled sections of the internal coiled body 136. A suture needle leading an anchoring suture 140 is shown being threaded through an outer periphery of the anterior ring segment.

FIG. 14A is a sectional view through the annuloplasty ring 130 indicating one possible path of the suture 140 through the ring. Namely, without the radially extended cuff 68, the surgeon must attempt to snag the suture needle 138 through the outer fabric cover 134, which is relatively thin. Inevitably, the needle 138 passes inward to some extent between coils of the coiled body 136 and potentially through a filler member 141. If the surgeon catches a number of suture needle 138 passes through the tightly coiled body 136, there results a high drag force on the array of sutures 140 as the annuloplasty ring 130 is parachuted down to the mitral annulus. Moreover, sutures 140 passed through the tightly wound portion of the coiled body 136 sometimes interferes with an even constriction of the annuloplasty ring, deforming the reduced diameter shape. This highlights a need to add a suture cuff to the outer periphery of the outer cover 134. More particularly, adding a suturing cuff permits passage of anchoring sutures therethrough instead of through the adjustable structural elements of the ring, helps maintain a generally annular shape of the ring during size adjustment, and minimizes dragging forces of sutures through the implant during delivery to the annulus.

FIG. 15 is a sectional view through an annuloplasty ring of the present application having an outer sewing cuff 68 as described above. One possible path of an anchoring suture through the ring is shown passing mostly through the sewing cuff 68, and at most through the outer cover 66. As mentioned, this sewing cuff configuration includes a ring of fabric 74 formed by a single layer that is attached by a series of stitches 76 along the upper and lower edges to the fabric cover 66. The fabric 74 is desirably a woven polyester velour cloth.

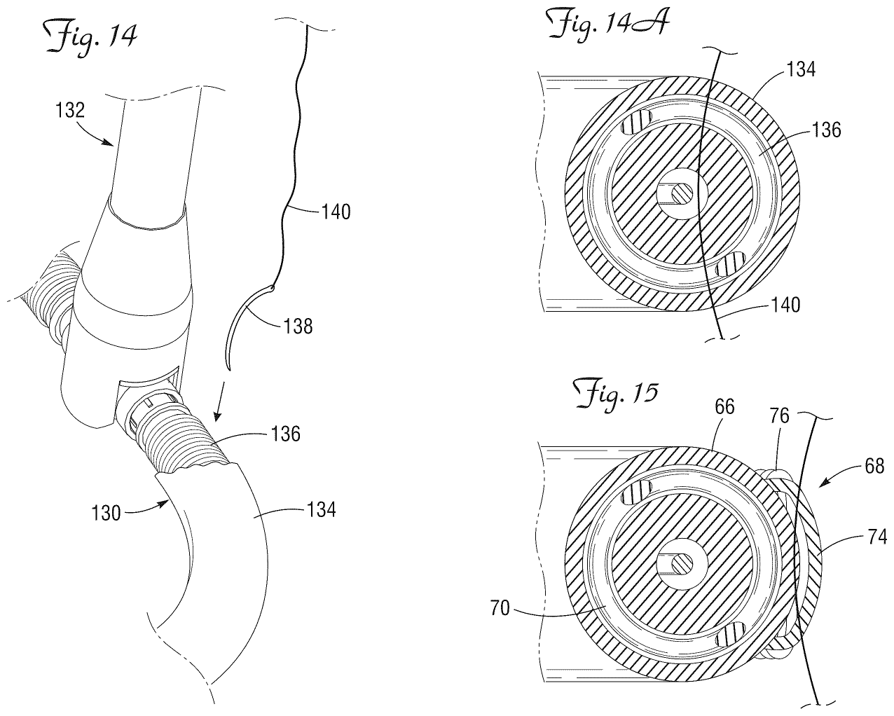

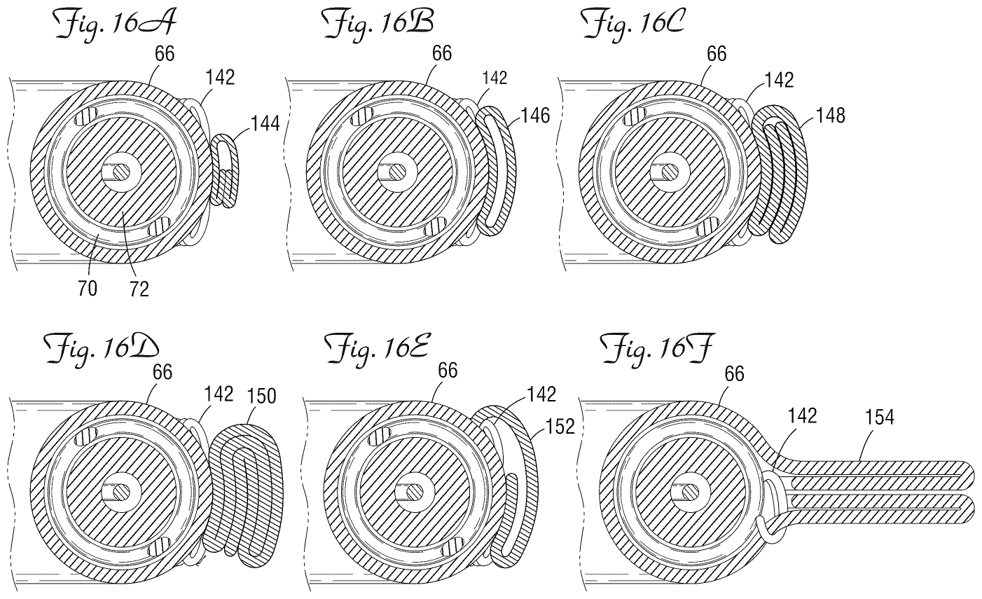

FIGS. 16A-16F are radial sectional views through alternative annuloplasty rings showing a variety of supplemental sewing cuffs that can be used. Each of the sewing cuffs are shown attached to a radially exterior periphery of an outer cover 66, and attached with two lines of stitching 142, as described above. First, FIG. 16A shows a cuff formed by a ring of folded cloth 144 which has a reduced axial dimension relative to the sewing cuff 68. The cloth 44 is rolled and tucked under at both of the bottom edges, and may be formed of a polymer fabric such as polyethylene terephthalate.

FIG. 16B shows another sewing cuff defined by a tubular ring of fabric 146, again attached by two rows of stitches 142. FIG. 16C shows a still further suture cuff 148 having a multiply folded strip of polyethylene terephthalate. More critically, the suture cuff 148 is folded upon itself so as to have four sequential radial layers. In FIG. 16D the sewing cuff 150 is also a strip of polyethylene terephthalate that is folded several times upon itself to result in six sequential radial layers. FIG. 16E shows a more loosely folded strip 152 forming the sewing cuff, the strip also desirably be formed of polyethylene terephthalate. FIG. 16F shows a sewing cuff 154 that is an extension of two peripheral edges of the outer cloth 66. More specifically, the two edges extend radially outward and are folded inward upon themselves and sutured together to form a flat sewing lip of sorts.

It should be understood that the various adjustable annuloplasty rings disclosed herein may be formed in a planar shape, as shown up to now, or in a three-dimensional saddle shape as are a number of prior art fixed-size annuloplasty rings. A saddle shape helps with restoring shape and dynamic motion of the annulus, and also helps provide in-plane stiffness with an out-of-plane flexibility to achieve and maintain the size reduction in the anterior-posterior dimension. Any of the annuloplasty rings described previously may be formed with a saddle shape, such as described below.

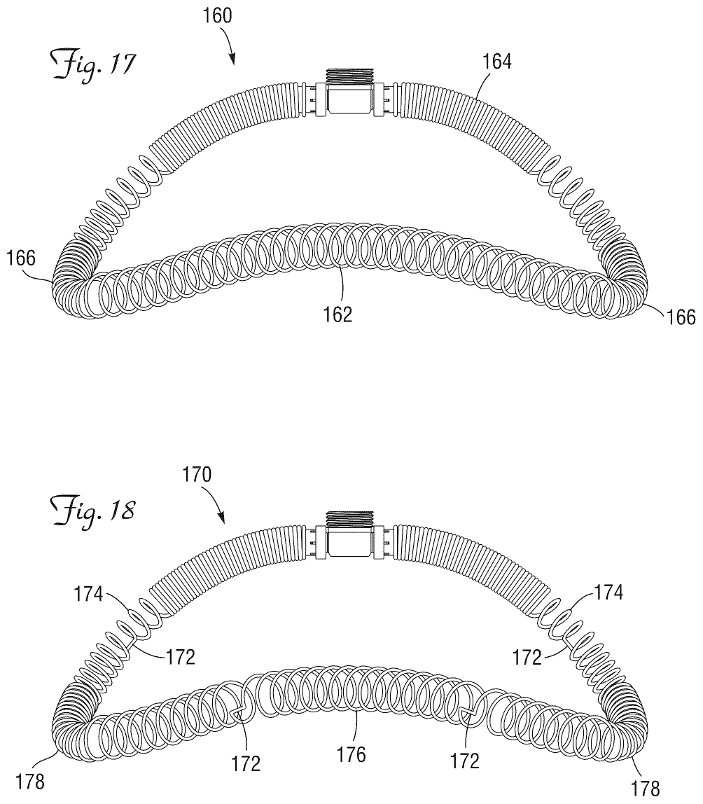

One particularly successful fixed-size ring of the prior art is available as the Physio II mitral annuloplasty ring from Edwards Lifesciences Corp. of Irvine, CA. FIG. 17 is a front elevational view of a saddle-shaped coiled body 160 in the shape of a Physio II ring that may be used with the adjustable annuloplasty rings of the present application. In particular, the coiled body 160 as an upward bow in the posterior segment 162, a higher upward bow in the anterior segment 164, and low points at the lateral side segments 166. The resulting shape is a lopsided saddle, somewhat resembling a lopsided Pringles brand potato chip. The coiled body 160 can be substituted for the coiled body 70 in the earlier-described adjustable annuloplasty ring 20, with the rest of the ring being unchanged. The coiled body 160 can again be formed of nitinol, which provides the stiffness and remodeling characteristics needed for mitral valve repair. The nitinol body 160 can be heat set into the desired saddle shape.

FIG. 18 is a front elevational view of an alternative saddle-shaped coiled body 170 having coiled regions of different spring rate. For example, a series of radially-extending junctions 172 may be provided to separate first segments 174, a second segment 176, and third segments 178. The first segments 174 are located adjacent to the tightly coiled anterior bow, the second segment 176 is centered on the posterior bow, and the two third segments 178 are located in between the first and second segments, roughly at the commissures of the mitral valve leaflets. The two pairs of first segments 174 and third segments 178 are desirably symmetric across a minor axis of the ring 170. Each of these helically coiled segments 172, 174, 178 may be configured with different spring rates, with the symmetric pairs of the first and third segments 174, 178 preferably being identical in spring rate to each other. This allows more control in regions around the annuloplasty ring 170 to address different pathologies. For example, the common condition of ischemic mitral regurgitation, or IMR, leads to varying stiffness around the mitral annulus. Providing relatively more flexible or stiffer segments around the ring periphery allows the surgeon to customize the ring for the particular pathology. For instance, one proposed coiled body 170 has more flexible third segments 178 adjacent the commissure regions of the mitral valve which are more susceptible to suture dehiscence. Additionally, one or more regions of the anterior or posterior side of the coiled body 170, such as the P3 segment (posterior side, closer to the posterior commissure PC), may be made stiffer to reinforce areas which are prone to prolapse.

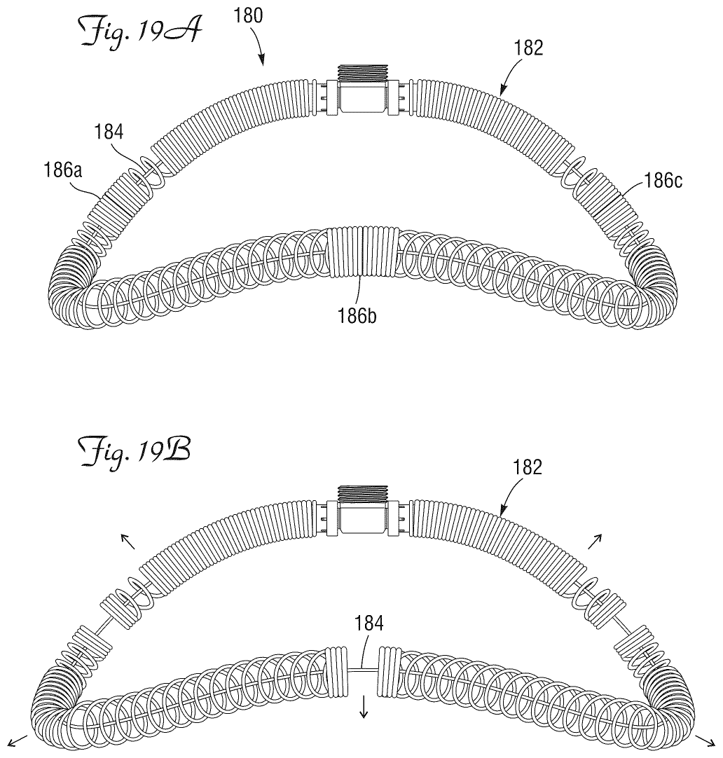

Another idea for the coiled body is to provide segments that are much more flexible to enable transient expansion during the systolic/diastolic cycle. FIG. 19A is a front elevational view of a saddle-shaped annuloplasty ring 180 with a coiled body 182, and FIG. 19B schematically illustrates expansion of the body during expansion of the annulus during the normal cardiac cycle. As before, the adjustable annuloplasty ring 180 has a cinching element 184 extending within the coiled body 182 and having a circumference that is controlled via a deployment system (not shown). The coiled body 182 features a series of expandable segments 186a, 186b, 186c distributed around its periphery which each include tightly wound coiled segments abutting one another. During the normal systolic/diastolic cycling, the tightly wound portions of the expandable segments 186a, 186b, 186c are allowed to spread apart, as seen in FIG. 19B.

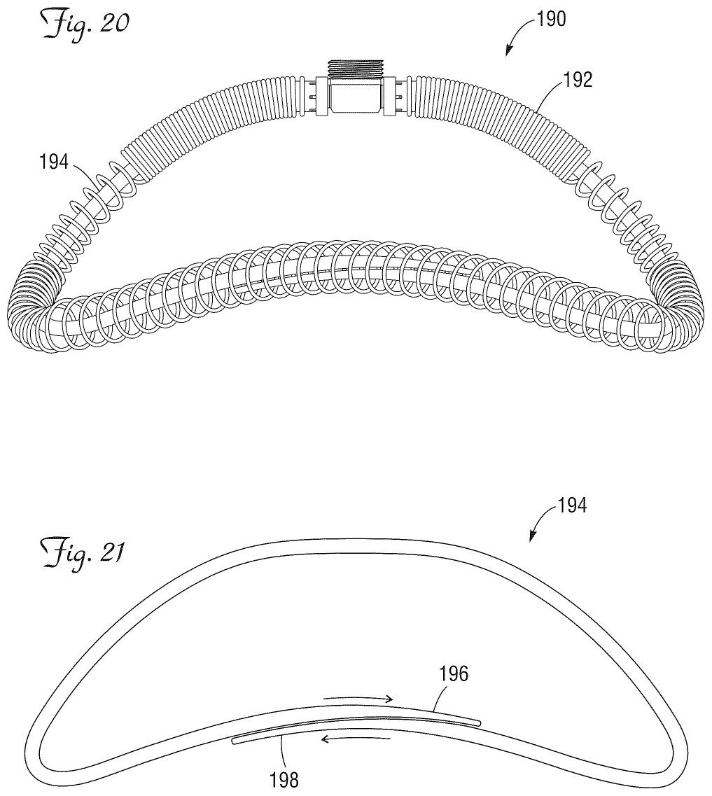

Another option is to have an adjustable ring that integrates design and performance characteristics of the Physio II, mentioned above, with another annuloplasty ring from Edwards Lifesciences available under the tradename Physio Flex. FIG. 20 is a front elevational view of another saddle-shaped annuloplasty ring 190 having a coiled body 192 and a size-adjustable remodeling core 194 positioned therethrough, and FIG. 21 is a front elevational view of the remodeling core. The ring 190 is shown without an outer suture-permeable cover for clarity. The remodeling core 194 has tapered free ends 196, 198 that overlap on a posterior side thereof and permit size adjustment of the ring 190.

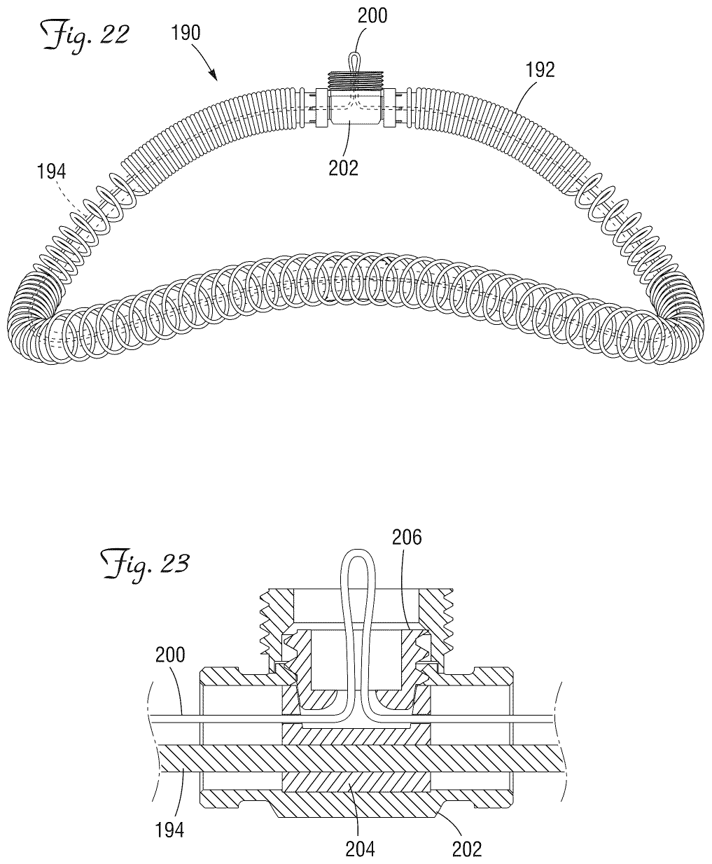

FIG. 22 is a view of the inner components of the saddle-shaped annuloplasty ring 190 with the addition of a cinching element 200, and FIG. 23 is a sectional view through a junction housing 202 thereof. The ring 190 is size-adjustable via the cinching element 200 in the same way as described above for the annuloplasty ring 20 and cinching element 60. That is, a deployment system (not shown) may be coupled to the junction housing 202 for advancing the ring 190 to the mitral annulus, and the deployment system may have an internal mechanism as described above for pulling the cinching element 200 up through the junction housing 202 to constrict the ring, and thus the annulus to which it is anchored.

As seen in FIG. 23, the solid anterior segment of the remodeling core 194 passes straight through a bore in a clamp member 204 held within the junction housing 202. The cinching element 200 extends through apertures on both lateral sides of the clamp member 204 and extends upward in a subloop through a central bore of a locking screw 206. The locking screw 206 is positioned below an upper port of the junction housing 202 and has external threads that engage internal threads therein. As with the locking screw 86 described above, the locking screw 206 may be advanced using a torque driver of a deployment system to clamp the cinching element 200 within the clamp member 204 once the proper size of the annuloplasty ring 190 is attained. The tapered free ends 196, 198 on the posterior side of the remodeling core 194 (see FIG. 21) slide relative to each other to permit cinching of the ring 190.

The remodeling core 194 may be formed of nitinol and heat set to the particular shape shown, though other materials such as stainless steel or a Co—Cr alloy may also be used. The remodeling core 194 is formed with the anterior and posterior bows, as shown, resulting in a saddle shape. The coiled body 192 need not be shape set to the saddle shape, and simply follows the contours of the core 194, which reduces manufacturing costs and may be stiffer and thus more effective in remodeling the annulus to a desired saddle shape.

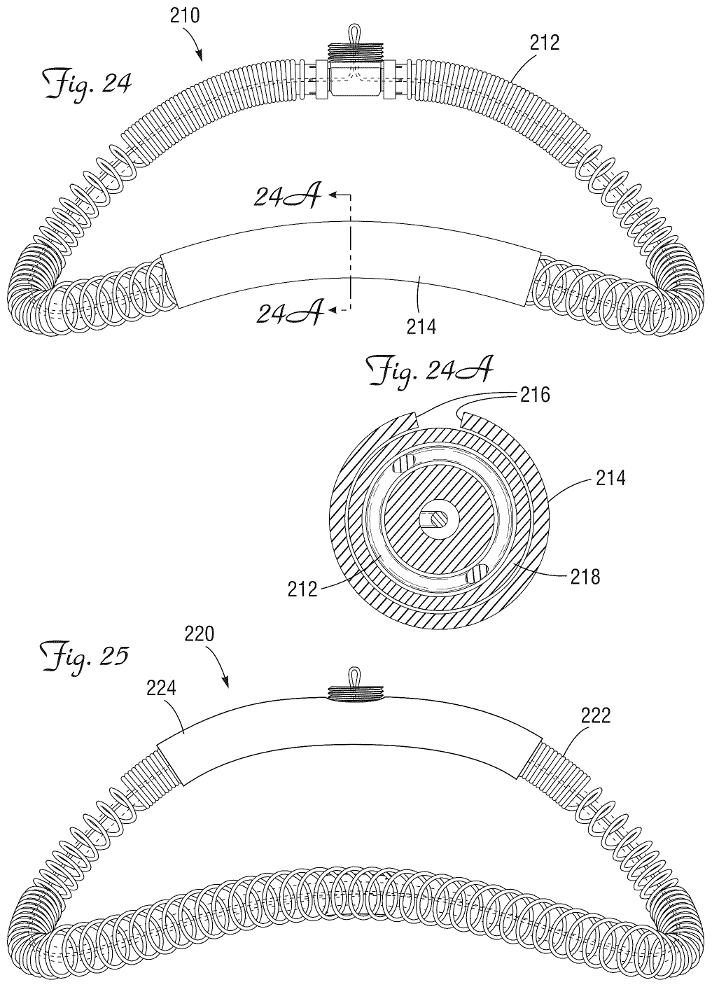

Another technique for shaping the otherwise flexible and adjustable annuloplasty rings is to add rigid shaped sleeves in strategic locations. For example, FIG. 24 is a front elevational view of inner components of a still further saddle-shaped annuloplasty ring 210 having a coiled body 212 and a shaped sleeve 214 surrounding a posterior segment to form an upward bow. FIG. 24A is a sectional view through the posterior segment thereof showing that the shaped sleeve 214 has an open slot 216 along an upper generatrix and closely surrounds a tubular friction-reducing member 218 surrounding the coiled body 212. The shaped sleeve 214 may be formed of a metal, such as titanium, Co—Cr or stainless steel, and defines the upward bow on the posterior side of the ring 210. The friction-reducing member 218 may be heat shrink polymer, silicone or cloth to limit metal-metal friction between the coiled body 212 and sleeve 214.

FIG. 25 is a front elevational view of inner components of another saddle-shaped annuloplasty ring 220 having a coiled body 222 surrounded by a shaped sleeve 224 on an anterior segment to form an upward bow. Again, the sleeve 224 may be formed of a metal, such as titanium, Co—Cr or stainless steel, and provides a rigid upward bow to the ring 220. In this embodiment, a central hole on the top of the sleeve 224 accommodates the junction housing which projects therethrough to receive a deployment shaft. Although not shown, a friction-reducing tube as described above may be introduced between the coiled body 222 and sleeve 224. Another alternative is to position the shaped sleeve 224 within the coiled body 222, in which case the friction-reducing member would be to the outside of the sleeve. Of course, the present application contemplates a ring with both a posterior sleeve 214 (FIG. 24) and an anterior sleeve 224 to provide two upward bows.

Many commercial annuloplasty rings for treating patients with mitral regurgitation have semi rigid cores or support bodies, such as the Physio II and Physio Flex rings from Edwards Lifesciences and the Memo 4D ring from LivaNova. Semi-rigid support bodies have the ability to remodel the annulus while preserving flexibility of movement. They typically include a saddle-shaped core to restore the shape and dynamic motion of the annulus. Occasionally, a sizing mismatch between the annulus and the semi rigid ring occurs once the heart is taken off cardiopulmonary bypass resulting in residual mitral regurgitation. The present application contemplates an improvement ring support body design which both maintains sufficient stiffness for annular remodeling while enabling adjustment of the ring size post-implant. Moreover, the support bodies disclosed herein present a reduced risk for suture dehiscence and maintain acceptable strain levels within the materials.

The adjustable continuous loop annuloplasty rings of the present application are designed to reduce mitral regurgitation in patients through a geometrical reduction of the mitral annulus anterior/posterior (A/P) dimension, as well as the Anterolateral posteromedial (AL-PM) dimension, thereby improving coaptation. The adjustable rings are primarily indicated for patients with degenerative mitral regurgitation. Providing a support body which is saddle shaped better matches the three-dimensional mitral annulus. In one embodiment, the support body is formed from a nitinol tube which is laser cut to have a specific pattern fine-tuned to meet the tensile strain requirements for nitinol, while still providing optimal stiffness to satisfy the annulus reconstruction or remodeling function. The rings thus promote the natural behavior of the mitral valve while also reducing the geometrical dimensions of the annulus. Further, the saddle shape of the ring combined with its semi-rigidity (e.g., semi-flexibility) helps alleviate the stress on the annulus, thereby reducing the risk of suture dehiscence and minimizing the rate of bleeding and stroke. Finally, the overall hoop flexibility of the semi-rigid nitinol rings is enough to accommodate a future valve-in-ring procedure, where an expandable valve is implanted within the ring.

Several proposed laser cut patterns in nitinol tubes are described herein. The designs were developed using finite element modeling to optimize the pattern for a given wall thickness to match the tensile strain levels established for nitinol, which designs could then be used to build prototypes for empirical testing. The laser cut patterns on the nitinol tubes are intended to achieve the following goals:
1. Maintain strains within the material property boundaries for nitinol if the annuloplasty ring is completely downsized.
2. Provide a stiffness which is comparable to annuloplasty rings formed of a cobalt-chromium alloy, for example, Elgiloy alloy (Elgiloy Specialty Metals).
3. Enable the support body to achieve a three-ring size reduction.
4. Enable the support body to achieve a one-ring size increase.
5. Provide variable tube stiffness along different regions of the support body for different patient etiologies.

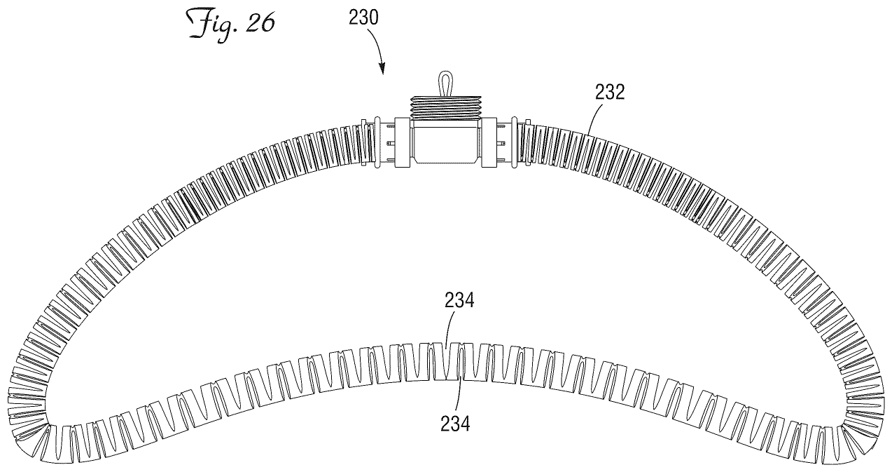

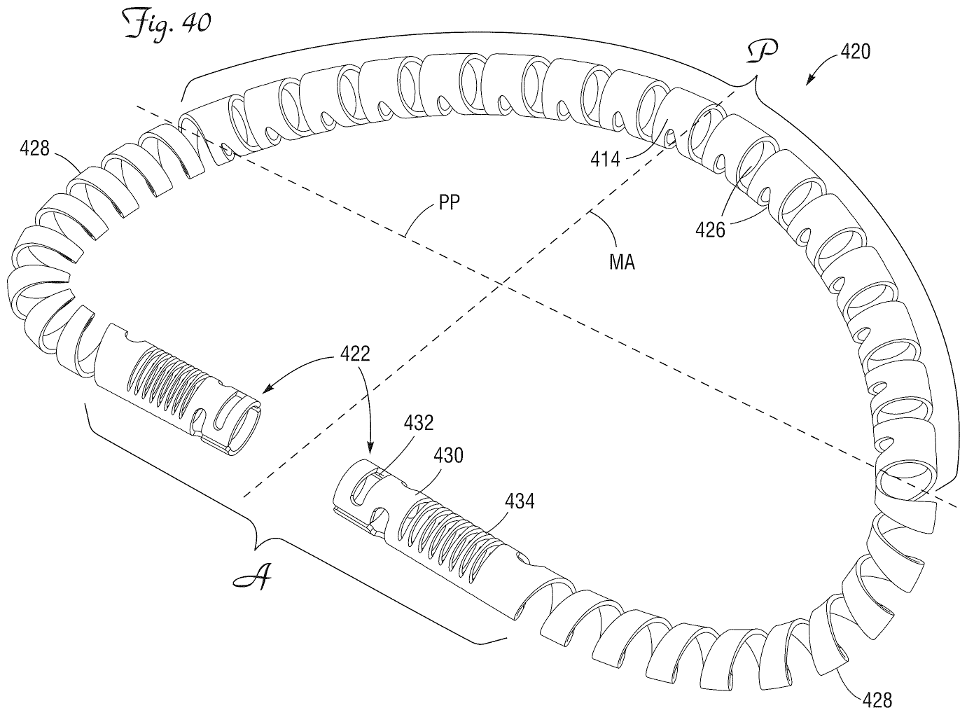

FIG. 26 is a front elevational view of an annuloplasty ring 230 having a saddle-shaped support body 232 formed by laser cutting a nitinol tube. A series of elliptical-shaped gaps 234 are cut into a tubular nitinol body, preferably before heat setting the body into the appropriate saddle-shape. The elliptical shape of the shape permits the design to be optimized to minimize high stress concentration areas. This construction enhances the ability to control the stiffness of the annuloplasty ring 230, while still enabling post-implant circumferential constriction as described herein. The width of the elliptical shaped gaps 234 around a circumference of the support body 232 may be varied to vary the post-implant flexibility around the ring. Again, it may be desirable to increase the flexibility at the lateral commissure regions while maintaining a relatively stiffer upward anterior and posterior curves to better support a diseased mitral valve prone to regurgitation. Furthermore, the wall thickness of the tubular blank used to form the support body may be modified to increase the overall stiffness if needed. Additional details of laser cut nitinol tubes used for the support body are shown in FIGS. 40-43 and described below.

The cinching elements disclosed herein are continuous loops, and thus the portion that sticks out the junction housing is relatively smooth and thus unobtrusive. However, sometimes the portion extending out of the junction housing is relatively long due to a large size reduction in the ring, which could cause problems and should be managed.

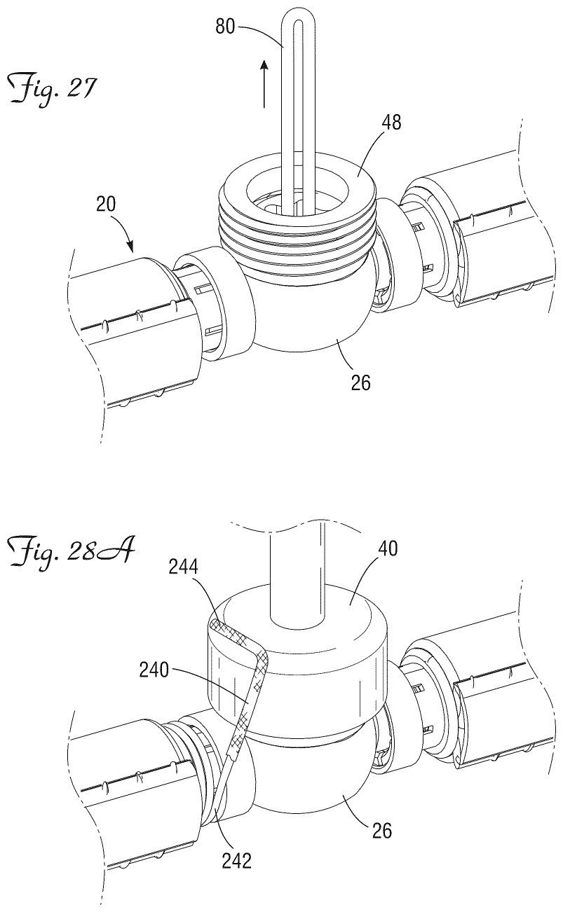

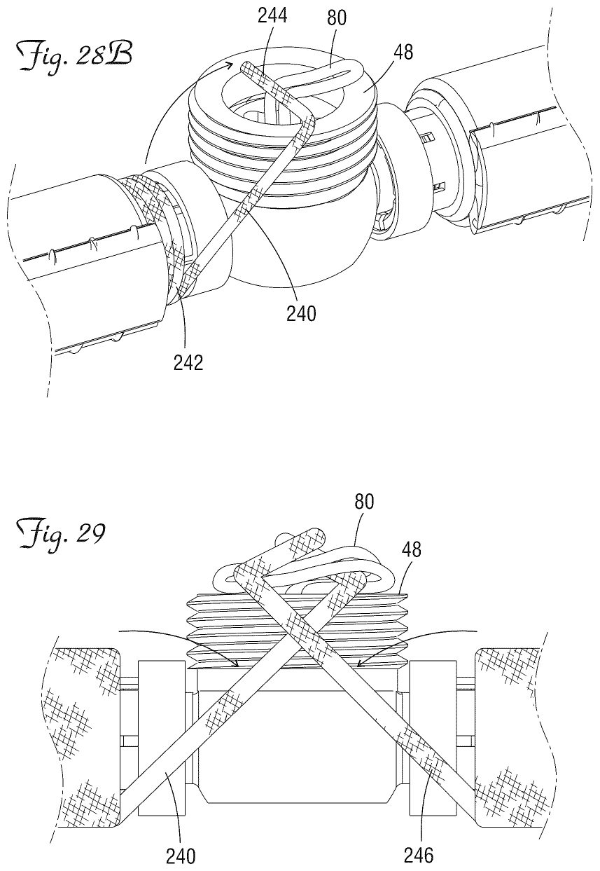

FIG. 27 is a perspective view of the junction housing 26 from which the subloop 80 has been retracted upward to cinch the annuloplasty ring 20. The portion of the subloop 80 extending above the upper port 48 can sometimes be relatively long. Consequently, FIGS. 28A and 28B illustrate a solution. FIG. 28A shows the junction housing 26 with a coupling member 40 of the deployment shaft attached. An exemplary spring-biased finger 240 is shown in contact with the exterior of the coupling member 40. In one embodiment, the finger 240 attaches around one of the side legs of the junction housing 26 so as to be spring biased toward the coupling member 40. A short perpendicular extension 244 of the finger 240 extends radially. The finger 240 may be covered with fabric, as shown.

FIG. 28B shows the inner coupling member 40 removed and deployment of the spring-biased finger 240 to fold the subloop 80 of the elongated cinching element against the junction housing 26. That is, the metallic subloop 80 is bent over so that it lies on top of the upper port 48. In the same manner, FIG. 29 is a side elevational view of an alternative configuration for folding the elongated cinching element 80 through the use of two spring-biased fingers 240, 246. The spring-biased fingers 240, 246 again may be covered with fabric, as shown.

The present application is mainly concerned with mechanisms for constricting the annuloplasty ring post-implant. However, sometimes the mitral valve that has been repaired fails completely, for instance due to excessive calcification, and must be replaced. Recent advances in heart valve replacements provide opportunities for implanting an expandable valve delivered percutaneously or transapically without stopping the heart and removing the native valve leaflets. Additionally, such expandable valves may be implanted within a previously implanted prosthetic heart valve, or within an annuloplasty ring. These are known as valve-in-valve and valve-in-ring procedures respectively. Often, such procedures are not available for smaller diameter and uses because of the presence of a relatively rigid previously implanted prosthetic valve or ring which reduces the total available orifice to an insufficient amount. However, some prior heart valves and annuloplasty rings are configured for expansion, and the present application contemplates adding such a feature to the rings disclosed herein.

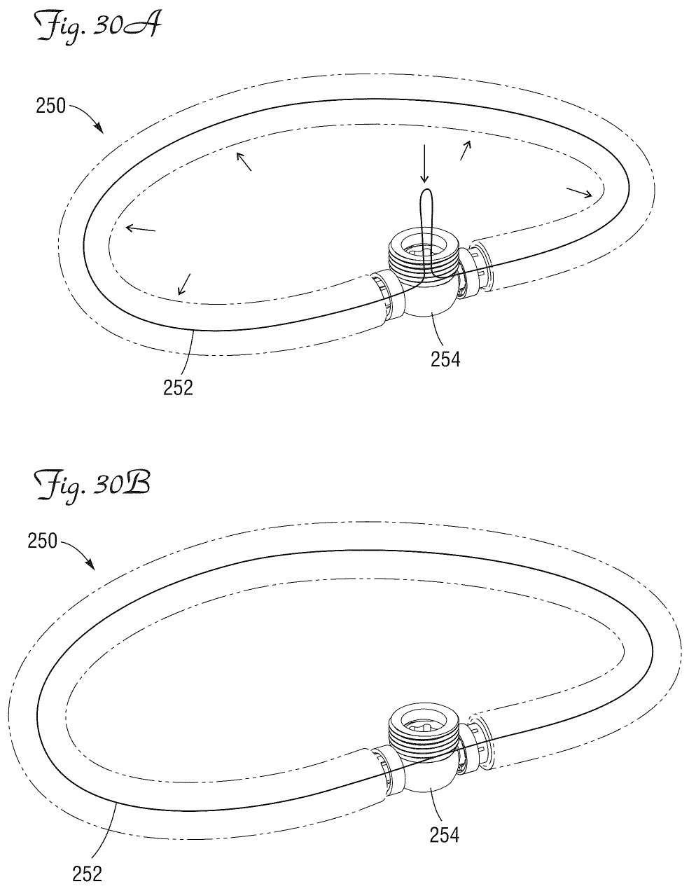

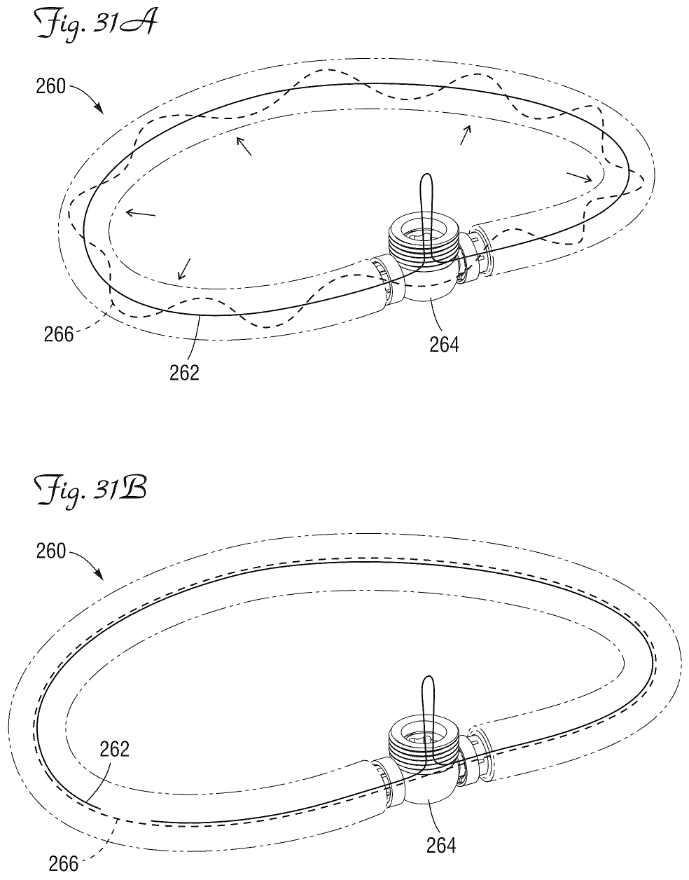

FIG. 30A is a schematic view of an adjustable annuloplasty ring 250 of the present application having a cinching element 252 and junction housing 254 that permit but limit expansion of the ring. The cinching element 252 extends around the periphery of the ring to and projects up through the junction housing 254, as has been described. Although the junction housing 254 contains a mechanism clamping the cinching element 252 and fixing a reduced size of the ring 250, the mechanism may be calibrated to resist expansion from natural cardiac cyclic forces but allow slippage and permit expansion from larger outward forces, such as from an expansion balloon.

Consequently, FIG. 30A shows outward forces being exerted on the ring 250, such as from an expansion balloon, which are large enough to pull the cinching element 252 through the clamping mechanism within the junction housing 254. The clamping mechanism within the junction housing 254 may be configured to have a spring element applying the locking force, therefore limiting the force by design. Ultimately, FIG. 30B is a view of the annuloplasty ring 250 after expansion showing the cinching element 252 stretched taut to its full circumference and limiting further expansion. That is, the subloop that previously extended up from the junction housing 254 has been pulled downward and now extends around the circumference of the ring 250. The material of the cinching element 252 is sufficient to withstand any further expansion from the balloon, and subsequently a valve-in-ring procedure may be performed to install a prosthetic valve within the ring 250.

FIG. 31A is a schematic view of another adjustable annuloplasty ring 260 having a cinching element 262 extending around the ring and up through a junction housing 264, and a secondary cord 266 within the ring to limit expansion thereof. The secondary cord 266 is shown in an undulating pattern around the periphery of the inside of the ring 260 so as to have excess length that serves no function during normal implantation, adjustment, and physiological function. During a valve-in-ring procedure, and in contrast with the version described above, the cinching element 262 is held firmly within the junction housing 264 but is configured to break upon application of expansion forces during a valve-in-ring procedure. FIG. 31B shows the annuloplasty ring 260 after expansion with the cinching element 262 broken and the secondary cord 266 expanded taut. The cinching element 262 may have a particular tensile strength which withstands normal physiological expansion forces, but breaks upon application of a larger force. Alternatively, the cinching element 262 could be provided with a weakened point which breaks upon application of the balloon expansion. The total length of the secondary cord 266 may be designed to allow installation of the largest prosthetic heart valve practical to be implanted within the particularly sized ring, and to have a sufficient tensile strength to avoid over-expansion. For example, the annuloplasty ring 260 may be sized for a relatively small annulus, such as 25 mm, with the total length of the secondary cord 266 being calibrated to be 27 mm. This accommodates installation of a prosthetic heart valve one size greater than the annulus.

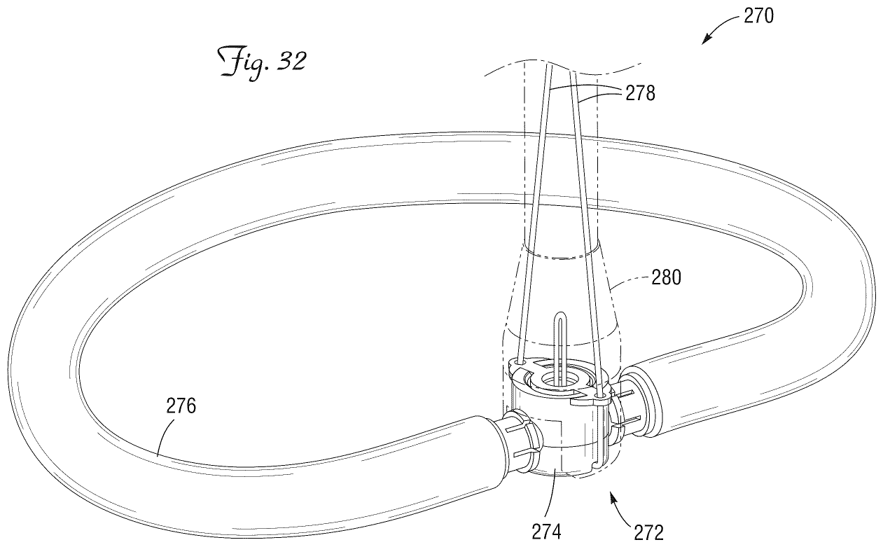

FIGS. 32-35 are various views of an annuloplasty ring 270 comprising an alternative junction housing 272 with certain modifications from junction housings described above. The modified junction housing 272 has a main body 274 constructed so as to be a molded part, and the annuloplasty ring 270 again has a peripheral rounded D-shaped body 276 that is fabric-covered for anchoring purposes. The annuloplasty ring body 276 may be constructed in any manner described herein. The new junction housing 272 enables a deployment system, represented by the distal end of the deployment shaft 280, to be secured to the housing via a quick-release harness 278.

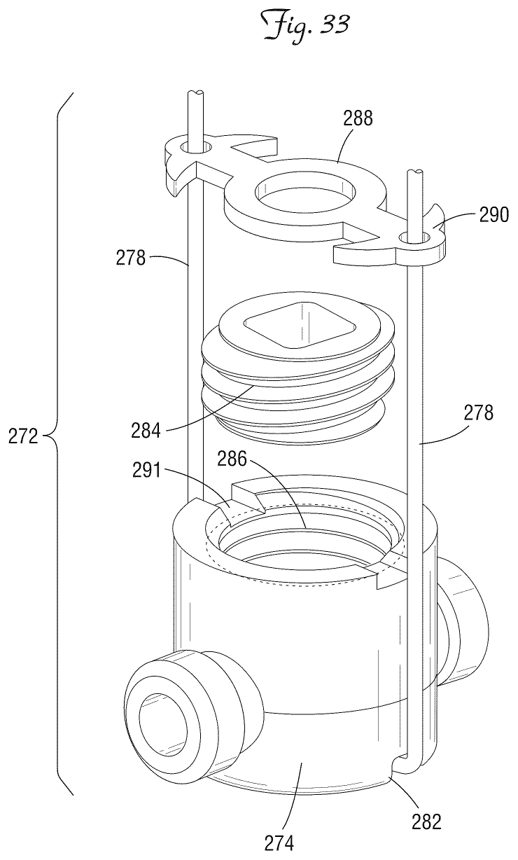

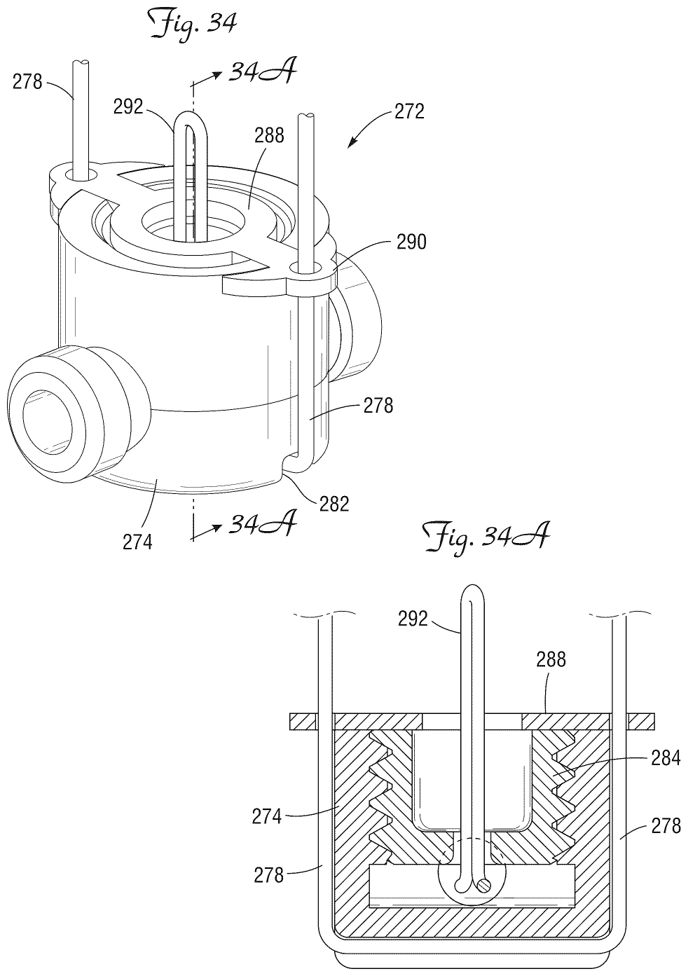

FIG. 33 shows the new junction housing 272 exploded. The housing 272 comprises the main body 274 having a radial channel 282 in a bottom surface that receives and retains the quick-release harness 278, which is in the form of an elongated strap or filament sized to loop under the main body. As seen in FIG. 32, the harness 278 extends proximally up through the deployment shaft 280 and eventually terminates in a control handle (not shown). The harness 278 thus retains the deployment shaft 280 in contact with the junction housing 272. Much like the earlier looped adjustment cable 58 described with respect to the first deployment system 24, the free ends of the harness 278 may be routed through a cleat and cutting guide assembly in the handle so that one strand may be severed and the entire harness then removed by pulling it out from under the junction housing 272. The quick-release harness 278 thus substitutes for the threaded connection in the first embodiment.

With reference to FIGS. 33-35, the junction housing 272 further has a locking screw 284 that mates with internal threads 286 within the main body 274, as before. A retention cap 288 having oppositely-extending wings 290 seats within slots 291 at the upper port of the main body 274 and may be ultrasonically-welded thereto to prevent the locking screw 284 from coming loose within the body. The wings 290 preferably have holes through which the strands of the harness 278 pass to help keep them aligned.

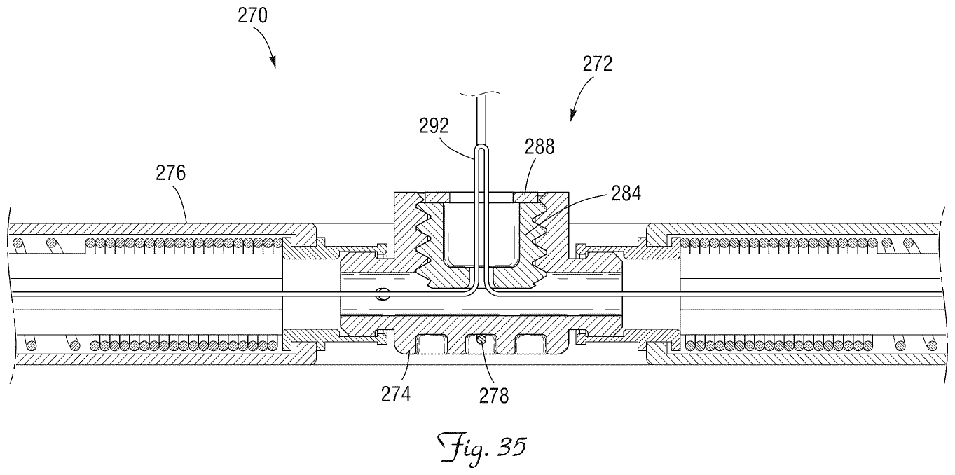

FIG. 35 shows the cross-section of the ring 270 with a cinching element 292 extending peripherally through an inner channel and emerging in a subloop through the junction housing 272. The deployment shaft 280 contains a tension mechanism such as described above which can pull the subloop 292 upward and thus cinch the ring 270 smaller. Likewise, the deployment shaft 280 also may incorporate a torque driver to advance the locking screw 284 and clamp the cinching element 292 in a final position within the junction housing 272. In all respects, the various mechanisms for cinching the ring may be as described above.

FIG. 36 is a schematic view of a size-adjustable annuloplasty ring 300 having an alternative cinching element 302 that extends internally around a periphery and through an anterior junction housing 304 in two directions. More particularly, the cinching element 302 may be a metallic wire (such as described above) with two terminal ends 306a, 306b which pass in opposite directions through a clamping mechanism (also such as described above) within the junction housing 304. Each of the terminal ends 306a, 306b connects to a pull suture 308a, 308b, preferably just outside of the junction housing 304. The pull sutures 308a, 308b extend internally in opposite directions alongside the cinching element 302 around the ring circumference and pass into the junction housing 304 from opposite directions. The pull sutures 308a, 308b are then directed upward in strands 310a, 310b through the upper port of the junction housing 304, through a deployment system 312, and eventually to a tensioning mechanism within a proximal control handle (not shown). Pulling on the strands 310a, 310b as indicated in FIG. 36 cinches the ring 300 smaller, as previously explained, and the cinching element 302 is clamped within the junction housing 304.

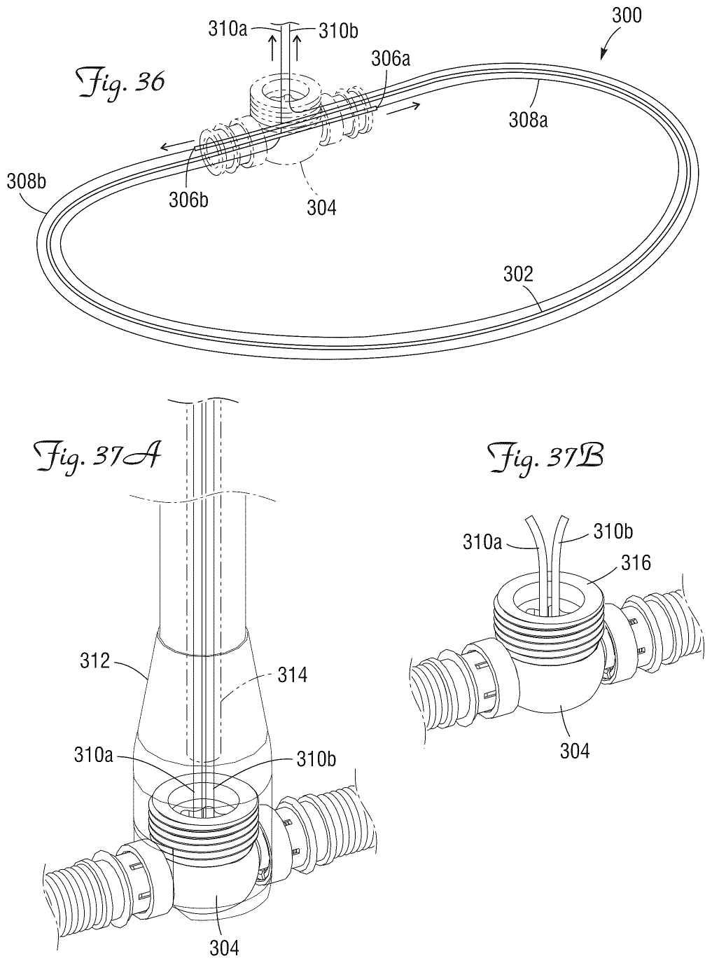

FIG. 37A shows a portion of the modified deployment system 312, and FIG. 37B is the junction housing 304 after cinching and severing the pull sutures 308a, 308b to leave just the cut off strands 310a, 310b. Instead of a looped cinching element as described elsewhere herein, the pull sutures 308a, 308b are severed by a modified deployment system 312 that incorporates a cutting tool, shown schematically as tube 314. Various ways to sever sutures are known in the art, and any could be implemented within the tube 314. Because the strands 310a, 310b are sutures, they do not present a significant risk of irritation to the mitral valve if the cut off ends are left hanging from the junction housing 304, as opposed to metallic ends. Moreover, the cinching element 302 that extends around the inside of the ring 300 and is clamped within the junction housing 304 is a metallic cable that is durable and resists creep.

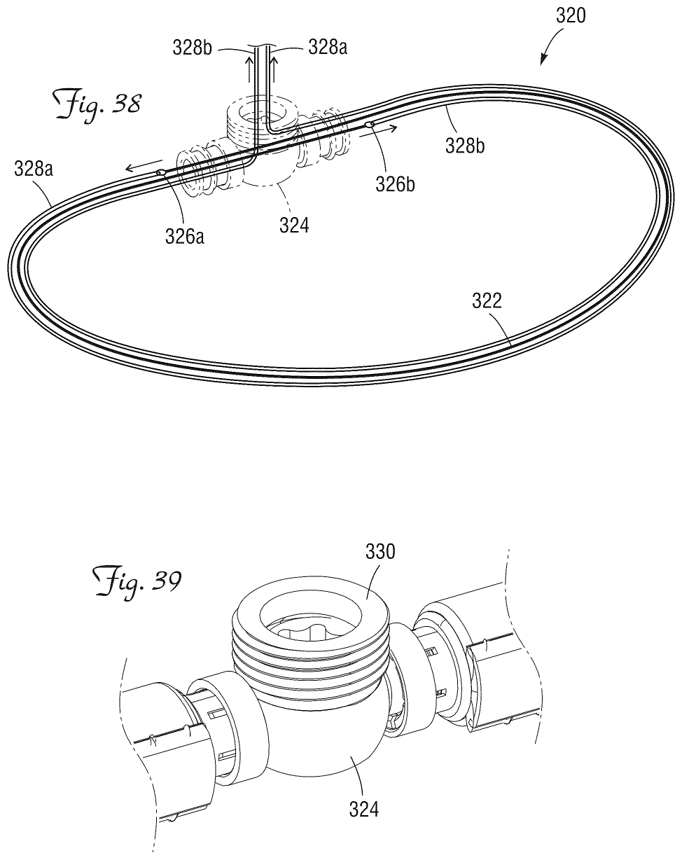

FIG. 38 is a schematic view of a further size-adjustable annuloplasty ring 320 having a cinching element 322, and FIG. 39 is a perspective view of a junction housing 324 therefor after cinching and removal of a pair of tension suture loops. The cinching element 322 once again extends internally around the ring circumference and through the anterior junction housing 324 in two directions, and may be a metallic wire as described above to prevent long-term creep. Terminal ends of the cinching element 322 have small rings 326a, 326b connected thereto. Two strands each of two tension suture loops 328a, 328b couple to the separate rings 326a, 326b. The suture loops 328a, 328b then extend in opposite directions alongside the cinching element 322 around the ring periphery and pass into the junction housing 324 from opposite directions. The tension suture loops 328a, 328b extend through the upper port 330 of the junction housing 324, through a deployment system (not shown) and eventually to a tensioning mechanism within a proximal control handle (not shown). Pulling on the suture loops 328a, 328b as indicated in FIG. 38 cinches the ring 320 smaller, as explained.

Once the ring 320 has been cinched to a desired size, the lengths of the cinching element 322 that extend through the junction housing 324 are clamped to prevent release. Subsequently, one strand of each of the two tension suture loops 328a, 328b is then released or cut at the control handle, allowing the entire loop to be pulled free from within the ring 320, through the junction housing 324 and also the deployment system. This leaves nothing projecting beyond the upper port 330 of the junction housing 324, as seen in FIG. 39.

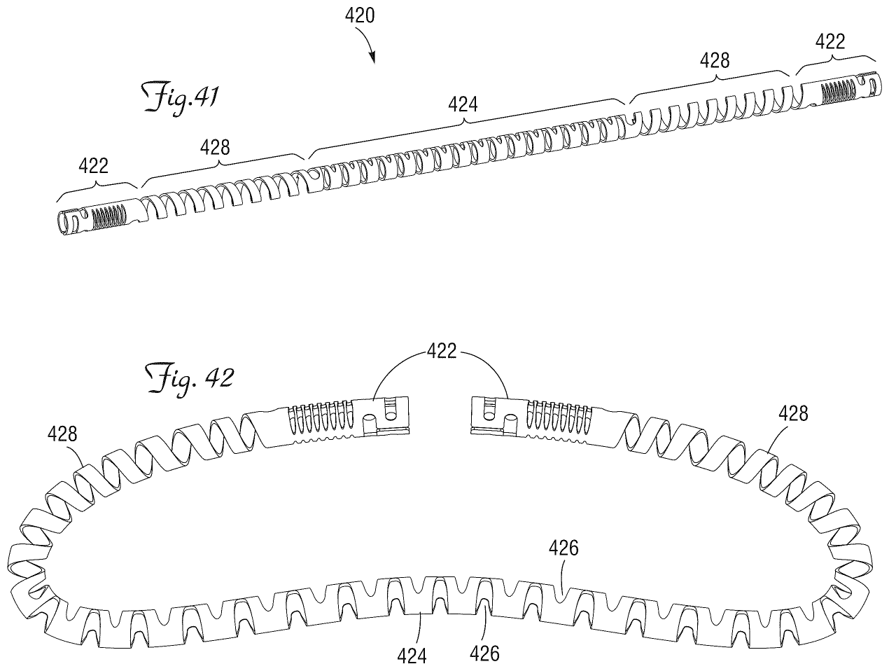

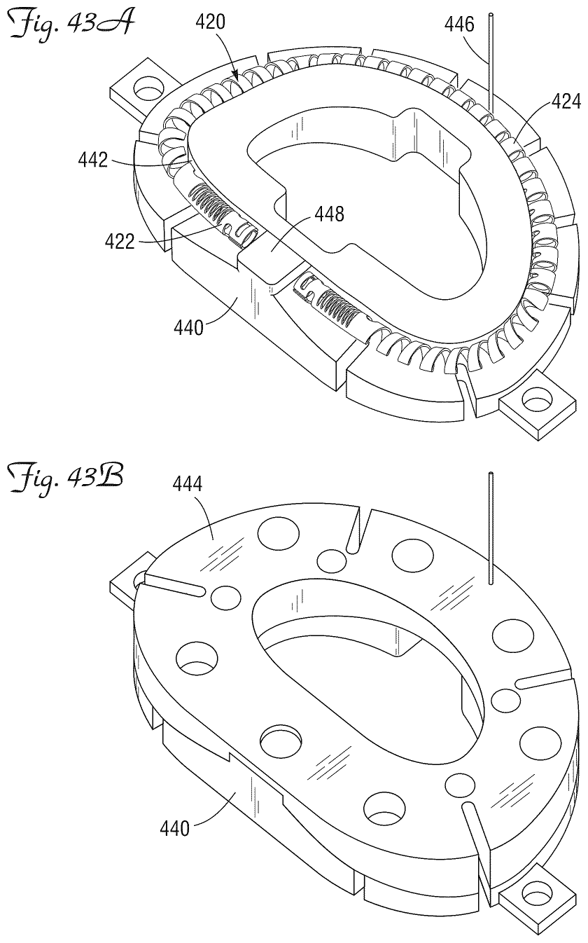

FIG. 40 is a perspective view of another saddle-shaped support body 420 for an annuloplasty ring formed by laser cutting a nitinol tube. FIG. 41 is a perspective view of the support body 420 in the form of a linear nitinol tube after having been laser cut, and FIG. 42 is a perspective view of the support body after the linear tube has been shape set. The support body 420 has a plan or atrial-side view which generally defines a rounded D-shape, with a relatively straight anterior segment A opposite a curved or convex posterior segment P. A posterior plane PP that denotes the ends of the posterior segment P is shown perpendicular to a minor axis MA across which the support body 420 is symmetric. The anterior segment A has a pair of generally solid-walled and linear free ends 422, while the posterior segment P is formed by a series of tubular segments 424 broken up by alternating inner and outer gaps 426. The anterior segment A is separated from the posterior segment P by lateral or side segments 428 formed by generally helical portions of the nitinol tube. The posterior segment P rises up to a midpoint, and the free ends 422 also rise up but not quite as much as on the posterior side.

In one embodiment, the alternating inner and outer gaps 426 between the tubular segments 424 in the posterior segment P described elliptical shapes of the same or different sizes. The depth of each of the gaps 426 alternates on opposite sides of the support body 420, with larger elliptical gaps 426 on the outer periphery and smaller ones on the inner periphery. A wall thickness of 0.25 mm is considered suitable.

The free ends 422 are each primarily a solid-walled tube 430 having a number of slots close to terminal ends for attaching to a junction housing, as seen below. A series of circumferential slits 434 on one or both top and bottom faces facilitates a small upward (atrial) bend in the anterior segment A to conform to the native rise in the anterior aspect.

The support body 420 is thus provided with areas of varying flexibility. The free ends 422 are relatively stiff, to both enable attachment to a junction housing and provide stiffness along the stiffer native anterior aspect. The side segments 428 on the other hand are highly flexible and notably compressible to facilitate the circumferential ring size reduction. Lastly, the posterior segment P is also somewhat flexible and compressible to facilitate ring size reduction, but not as flexible as the side segments 428 in order to maintain the upward bow or saddle and maintain conformance with the native posterior aspect. Looked at another way, the side segments 428 are relatively compressible/expandable in all directions, while the posterior segment P is somewhat compressible/expandable but stiffer in the plane of the support body 420 than in other directions, such as up and down.

The support body 420 is laser cut when in the linear tubular shape of FIG. 41, and then converted to the three-dimensional ring shape of FIG. 42 by heat treating/setting the tube to shape set it. FIG. 43A is a perspective view of a lower portion 440 of a shape setting mandrel having the laser cut support body 420 positioned thereon, and FIG. 43B is a mandrel assembly including an upper portion 444 that sandwiches the laser cut support body for shape setting. The lower portion defines a shaped groove 442 size to closely receive the tubular support body 420, which may be manually flexed and placed into the groove due to the flexibility of the laser cut nitinol. The upper portion 444 may have a groove similar but mirror image of the groove 442. The upper portion 444 of the mandrel assembly is then placed over the lower portion 440, with a mandrel alignment pin 446 helping to hold the support body 420 in place within the grooves 442. The lower portion 440 also includes an upwardly-projecting block 448 on the anterior side which both separates the free ends 422 of the support body 420 and fits within a similar-sized recess (not shown) in the upper portion 444.

Once the support body 420 is held within the grooves 442 with the upper and lower portions 440, 444 held together, heat is applied for a predetermined amount of time to change the crystalline structure of the nitinol in the support body and set its shape. In one example, the shape setting parameters are 260° C. (500° F.) for five minutes, although these may change depending on the particular alloy of nitinol used as well as the wall thickness. Once shape set, the support body 420 is electropolished to remove any sharp burrs or other roughness/particles. The tensile strain within the nitinol material of the support body 420 formed in accordance with the patterns and shape setting herein is not expected to exceed 0.07%.

It should be apparent that the individual features of the various size-adjustable annuloplasty rings described herein may be combined, if not explicitly stated. For instance, the cinching element 322 and tension suture loops 328a, 328b described with respect to FIGS. 38-39 may easily be incorporated into the annuloplasty ring 20 of the first embodiment, while the various suture cuffs shown in FIGS. 15-16 could be used on any of the rings. It is therefore an axiom that any combination of features is contemplated short of them being redundant, mutually exclusive and/or physically impossible, and the claims presented below should not be viewed otherwise.

While the foregoing is a complete description of the preferred examples, various alternatives, modifications, and equivalents may be used. Moreover, it will be obvious that certain other modifications may be practiced within the scope of the appended claims.

What is claimed is:

1. An annuloplasty ring and shape adjustment system, comprising:
an annuloplasty ring defining a continuous peripheral shape around a central aperture and a central axis, the annuloplasty ring having a contractible inner body defining a lumen extending therethrough, wherein the inner body extends around the peripheral shape and has two ends connected to each other by a junction housing, the annuloplasty ring further including a flexible non-extensible cinching element formed in a complete loop, the cinching element passing through the inner body lumen and into the junction housing, wherein the junction housing has an inner cavity with three openings—first and second aligned openings in communication with the inner body lumen at the two ends of the inner body, and a third opening at an upper port facing axially up from the housing—wherein the cinching element has a main portion generally in a plane of the inner body and a subloop that projects perpendicular to the main portion through the upper port, and wherein the inner body has a relaxed implant shape and a smaller, contracted shape when the cinching element is placed under tension.

2. The system of claim 1, wherein the junction housing has external threads for attachment of a delivery shaft thereto.

3. The system of claim 1, wherein the annuloplasty ring further includes a locking member within the junction housing configured to clamp onto the cinching element and maintain the cinching element under tension around the inner body.

4. The system of claim 3, wherein the locking member comprises a locking screw positioned in a vertical channel of the junction housing aligned with the upper port, the locking screw having external threads which engage internal threads defined within the vertical channel, and the locking screw having a non-circular internal throughbore that receives the subloop of the cinching element, wherein rotational advancement of the locking screw down into the vertical channel eventually clamps divergent portions of the cinching element against the inner cavity of the junction housing.

5. The system of claim 1, wherein the cinching element is metallic.

6. The system of claim 5, wherein the cinching element is a cobalt chromium alloy.

7. The system of claim 1, wherein the inner body is formed of a metallic tube having V-shaped gaps formed therein to permit contraction.

8. The system of claim 7, wherein the inner body is heat set into a saddle shape.

9. The system of claim 1, further including a hollow compressible filler member extending within the inner body lumen and through which the cinching element extends.

10. The system of claim 1, further including a tubular fabric cover surrounding the inner body and having a sewing cuff of a ring of fabric attached to an outer periphery of the fabric cover.

11. An annuloplasty ring and shape adjustment system, comprising:
an annuloplasty ring defining a continuous peripheral shape around a central aperture and a central axis, the annuloplasty ring having a contractible inner body defining a lumen extending therethrough, wherein the inner body extends around the peripheral shape and has two ends connected to each other by a junction housing, the annuloplasty ring further including a flexible cinching element formed in a continuous loop, the cinching element passing through the inner body lumen and into the junction housing, wherein the cinching element forms a subloop through a port formed in the junction housing, and wherein the inner body has a relaxed implant shape and a smaller, contracted shape when the cinching element is placed under tension;
a deployment system including an elongated deployment shaft having a distal end configured to attach to the port of the junction housing, and a proximal control handle, the control handle having an adjustment cable connected to a tension actuator configured to pull the adjustment cable in a proximal direction, wherein the adjustment cable has a length sufficient to extend through a lumen in the elongated shaft and loop through the subloop, wherein actuation of the tension actuator pulls the adjustment cable and in turn places the cinching element under tension to convert the inner body from the relaxed implant shape toward the smaller, contracted shape.

12. The system of claim 11, wherein the annuloplasty ring further includes a locking member within the junction housing configured to clamp onto the cinching element and maintain the cinching element under tension around the inner body, and the control handle has a locking actuator connected to an elongated torque shaft configured to slide axially and rotate concentrically within the deployment shaft, the torque shaft having a distal non-circular torque driver shaped to mate with a similarly-sized and shaped opening in the locking member, wherein rotation of the torque shaft causes the locking member to clamp onto the cinching element.

13. The system of claim 12, wherein the locking member comprises a locking screw positioned in a vertical channel of the junction housing aligned with the port, the locking screw having external threads which engage internal threads defined within the vertical channel, and the locking screw having a throughbore that receives the subloop of the cinching element, the torque shaft and torque driver being hollow to receive the subloop and the adjustment cable passes through the torque shaft to loop through the subloop, wherein rotational advancement of the locking screw down into the vertical channel eventually clamps divergent portions of the cinching element against the inner cavity of the junction housing.

14. The system of claim 12, wherein the tension actuator and locking actuator are wheels positioned within a housing of the control handle and mounted to rotate about a longitudinal axis of the housing, the wheels being exposed through windows formed in the housing and each having a locking collar associated therewith with a tab extending to the exterior of the housing, the tabs being arranged to toggle between permitting and preventing rotation of a respective wheel.

15. The system of claim 14, wherein the wheels each have a scalloped or uneven outer surface to facilitate rotation.

16. The system of claim 11, wherein the junction housing has external threads for attachment of the delivery system thereto, and the control handle has an attachment actuator connected to an elongated attachment shaft configured to slide axially and rotate concentrically within the deployment shaft, the attachment shaft having a distal end with internal threads for mating with the external threads on the junction housing, wherein rotation of the attachment shaft enables attachment and detachment of the delivery system from the annuloplasty ring.

17. The system of claim 11, wherein the delivery system has a quick-release harness in the form of a loop of flexible material that extends from the control handle and around the junction housing, wherein one end of the loop of flexible material is severable within the control handle to enable the detachment of the delivery system from the annuloplasty ring.

18. The system of claim 11, wherein the cinching element is metallic.

19. The system of claim 11, wherein the inner body is formed of a metallic tube having V-shaped gaps formed therein to permit contraction.

20. The system of claim 19, wherein the inner body is heat set into a saddle shape.

21. The system of claim 19, wherein the annuloplasty ring also includes a hollow compressible filler member extending within the inner body lumen and through which the cinching element extends, and a tubular fabric cover surrounding the inner body and having a sewing cuff of a ring of fabric attached to an outer periphery of the fabric cover.
